(19) United States
(12) Patent Application Publication
(10) Pub. No.: US 2015/0233930 A1
(43) Pub. Date: Aug. 20, 2015

(54) METHODS AND COMPOSITIONS EMPLOYING SECRETED PROTEINS THAT REFLECT AUTOPHAGY DYNAMICS WITHIN TUMOR CELLS

(71) Applicants: The Wistar Institute of Anatomy and Biology, Philadelphia, PA (US); The Trustees of the University of Pennsylvania, Philadelphia, PA (US)

(72) Inventors: David W. Speicher, Berwyn, PA (US); Adam Kraya, Garnet Valley, PA (US); Ravi K. Amaravadi, Media, PA (US)

(21) Appl. No.: 14/615,635

(22) Filed: Feb. 6, 2015

Related U.S. Application Data
(60) Provisional application No. 61/940,181, filed on Feb. 14, 2014.

(51) Int. Cl. G01N 33/574; C12Q 1/68

(52) U.S. Cl. CPC G01N 33/57484; C12Q 1/6886; C12Q 2600/158

(57) ABSTRACT

A diagnostic reagent or device for determining the autophagy levels of cancer cells comprises a ligand capable of specifically complexing with, binding to, or quantitatively detecting or identifying a biomarker selected from IL-8, IL-1β, DKK-3, FAM-3C and/or LIF, or an isoform, pro-form, modified molecular form including posttranslational modification, or unique peptide fragment or nucleic acid fragment thereof. Another reagent contains ligands to two or more of these biomarkers. Optionally, such reagent or device includes a signaling molecule and/or a substrate on which the ligand is immobilized. Other reagents are useful in methods of measuring autophagy of cancer cells, e.g., melanoma and for monitoring cancer progression, chemoresistance, or predicting likely candidates for treatment with autophagy inhibitors.

### CROSS-REFERENCE TO RELATED APPLICATIONS

This application claims the benefit of the priority of US Provisional Patent Application No. 61/940,181, filed Feb. 14, 2014, which application is incorporated herein by reference in its entirety.

### STATEMENT REGARDING FEDERALLY SPONSORED RESEARCH OR DEVELOPMENT

This invention was made with government support under Grant Nos. 5T32HG000046-15, 1K23CA120862, R01 CA109618 and CA010815, awarded by the National Institutes of Health. The government has certain rights in this invention.

### BACKGROUND OF THE INVENTION

Autophagy¹ is a catabolic process that sequesters damaged organelles and protein aggregates for degradation. Regulated by a vast intracellular signaling network, autophagy occurs at basal levels in virtually all cells to perform its homeostatic functions and adaptively responds to environmental stresses to confer cell survival². Autophagy initiates with the nucleation of an autophagic vesicle, generated from the surface of source membranes by class III phosphoinositide-3-kinase (PI3K) and Beclin 1³. Light chain protein 3 (LC3) integrates into the autophagic vesicle bilayer and is conjugated to phosphatidyl-ethanolamine by ubiquitin ligase-like protein conjugation complexes. Fusion of autophagic vesicles (AV) with lysosomes results in the degradation of vesicle cargo by acid hydrolases. Autophagic vesicle components that are degraded are recycled to fuel further growth via multiple transporters on the surface of the autophagolysosome⁴. Although autophagy operates at low basal levels to maintain cellular homeostasis in nearly all cells; metabolic, hypoxic, and therapeutic stresses can provoke a series of adaptive responses that amplify autophagic activity in cancer⁵.

Autophagy plays important roles in tumor survival and resistance to targeted therapies for some cancers and is a potential cancer therapeutic target. Autophagy is a common mechanism of chemoresistance, an emerging problem in the treatment of advanced cancers. In melanoma, persistently high levels of autophagy in advanced tumors along with enhanced efficacy of chemotherapeutics upon autophagy inhibition suggest that autophagy is a targetable resistance mechanism. Recent evidence suggests that autophagy serves a pro-tumorigenic role in established tumors, facilitating cell survival in the face of microenvironmental stress in the form of hypoxia, nutrient deprivation, or cytotoxic drugs⁵. Autophagy may be especially relevant to melanoma aggressiveness, since the melanocyte already has high levels of autophagy present in order to produce melanin⁶, and melanomas frequently have RAF or RAS mutations that may necessitate higher levels of autophagy to survive oncogene-induced stress^{7,8}. There is emerging evidence that autophagy is not only involved in the intracellular degradation of damaged proteins, but also plays an important role in non-classical protein secretion¹¹⁻¹³.

In preclinical models of melanoma, inhibition of autophagy with either hydroxychloroquine or targeted knockdown of the essential autophagy gene ATG5 was also found to augment the cytotoxic effects of alkylating agents in tumor cells, suggesting a role for autophagy in survival¹. Pretreatment patient melanoma tumor samples revealed a striking level of heterogeneity in autophagy levels^{1,5}, and high autophagy was associated with tumor cell survival, chemotherapeutic resistance, and metastasis. Low autophagy levels predicted better therapeutic outcomes in chemotherapy based trials. High levels of autophagy in pretreatment tumors could predict those patients that may exhibit the best response to autophagy inhibitors.

Efforts to inhibit autophagy in clinical trials have been hampered by sub-optimal methods to quantitatively measure tumor autophagy levels, the lack of noninvasive assays that reflect autophagy dynamics within the cancer cell, and a poor understanding of the role of autophagy-mediated secretion on the tumor microenvironment. Currently, there are no predictive biomarkers or quantitative preclinical assays that reproducibly monitor autophagy and can be easily translated into clinical predictive assays. The lack of such assays impedes the development of autophagy inhibitors in the clinic⁹. Electron microscopy allows for direct quantitation of autophagic vesicles, but measurements are subjective and the procedure is laborious and costly. Furthermore, it is often performed on surrogate tissues rather than on tumor tissue, and such measurements may not always reflect the autophagy dynamics of the tumor microenvironment. Assays relying on the detection of LC3 provide insight into the prevalence of mature autophagic vesicles, but these methods are semi-quantitative and not easily translated into monitoring clinical samples, as LC3 expression is difficult to detect in tissue¹⁰.

There is also a striking difference in the autophagic response when cells grown in 2D cultures are compared with cells growing in the tumor microenvironment. 3D cell culture appears to more faithfully reproduce autophagy dynamics found in the tumor microenvironment compared to traditional 2D culture. However, quantitative analytical methods to confirm these qualitative observations have not yet been developed.

### SUMMARY OF THE INVENTION

In one aspect, a diagnostic reagent or device comprises one or more ligands, each ligand capable of specifically complexing with, binding to, or quantitatively detecting or identifying one biomarker selected from a biologically diverse panel of novel predictive biomarkers of tumor cell autophagy. Such a panel of biomarkers includes

(a) IL-8 or an isoform, pro-form, modified molecular form, posttranslational modification, or unique peptide fragment or unique nucleic acid fragment thereof;

(b) IL-1β or an isoform, pro-form, modified molecular form, posttranslational modification, or unique peptide fragment or unique nucleic acid fragment thereof;

(c) DKK-3 or an isoform, pro-form, modified molecular form, posttranslational modification, or unique peptide fragment or unique nucleic acid fragment thereof;

(d) FAM-3C or an isoform, pro-form, modified molecular form, posttranslational modification, or unique peptide fragment or unique nucleic acid fragment thereof;

(e) LIF or an isoform, pro-form, modified molecular form, posttranslational modification, or unique peptide fragment or unique nucleic acid fragment thereof; and/or

(f) an isoform, pro-form, modified molecular form, posttranslational modification, or unique peptide fragment or unique nucleic acid fragment thereof, proteins in the same biomarker family or expressed from a related gene, having at least 20% sequence homology or sequence identity with any biomarker (a) to (e). These biomarker expression or activity levels reflect intracellular autophagy dynamics. In still other embodiments, the panel of biomarkers includes other biomarkers or ligands to the biomarkers identified below.

In another aspect, the reagent, kit or device described herein comprises a panel or microarray of such ligands or their biomarker targets.

In another aspect, a method is provided for identifying autophagy level of a cell in a mammalian subject's biological sample, or identifying likely chemoresistant cancers or identifying subjects that are likely candidates for treatment with autophagy inhibitors. These methods employ the reagents, kits or devices described herein which can identify the presence or changes in expression or activity levels of target biomarkers in a biological sample, particularly blood, of a mammalian subject. A significant modulation in expression or activity level of a biomarker described herein in the subject's biological sample relative to a selected reference standard correlates with an increase or decrease in the autophagy level in the subject's cells.

In another aspect, an assay method for predicting the sensitivity of a subject with cancer to treatment with an autophagy inhibitor is provided.

In yet a further aspect, a method for treating a subject with melanoma comprises (a) contacting a biological sample obtained from a test subject with a diagnostic reagent or device described herein; (b) quantitatively measuring tumor autophagy levels in the subject by detecting or measuring in the sample or from a protein level profile generated from the sample, the protein levels of at least one biomarker of claim 1; (c) comparing the protein level of the biomarkers in the subject's sample with the level of the same biomarker in a healthy reference standard or in a reference standard of subjects with non-chemoresistant melanoma; and (d) treating said subject with an autophagy inhibitor, when the biomarker expression levels in the subject's tumor sample are increased over that of the reference standard.

Other aspects and advantages of these compositions and methods are described further in the following detailed description of the preferred embodiments thereof

### BRIEF DESCRIPTION OF THE DRAWINGS

FIGS. 1A-1D demonstrate the autophagic capacity of paired melanoma cell lines and cellular response to growth conditions. FIG. 1A are representative electron micrographs and box plots of mean AV/cell for WM973 and 1205Lu cells grown in complete medium in two dimensional cell culture; *p<0.05 (N=6). FIG. 1B show immunoblotting of lysates from cells treated as indicated. Rapa: rapamycin; HCQ: hydroxychloroquine. FIG. 1C is a schematic of 3D cell culture spheroid model illustrating secretome sample acquisition from the overlaying media and interstitial fluid (media in the collagen matrix) compartments. FIG. 1D show Live/Dead Assay in three dimensional spheroids grown in a collagen matrix after exposure for 24 hours to either serum-free media or media with 1% fetal bovine serum. Light color (live)—Calcein AM; Darker spots (dead)—Propidium Iodide.

FIGS. 2A-2E illustrate the characterization of differentially secreted proteins by low and high autophagy melanoma cells. FIG. 2A is a distribution of significantly elevated proteins (2-fold or greater) in either 1205Lu or WM793. FIG. 2B is a classification of differentially secreted proteins by GO terms for cellular component using STRAP. Proteins may be assigned to multiple cellular component categories; percentages are computed as fraction of total assignments. FIG. 2C shows the classification of differentially secreted proteins by GO terms for biological process. Proteins may be assigned in multiple biological process categories; percentages are computed as fraction of total assignments. FIG. 2D illustrate the top-ranking subnetwork identified by shortest paths analysis and betweenness-centrality clustering based on functional relations from STRING. FIG. 2E shows a second-ranked functional subnetwork. Proteins noted with a box are differentially secreted proteins identified herein.

FIGS. 3A-3F demonstrate the measurement of candidate biomarkers of high autophagy in a panel of high and low autophagy melanoma cell lines. FIG. 3A top panel shows the LC3 immunoblotting of total cell lysates for biological duplicates of WM9, WM1346, WM1361A, WM164, WM1366, and A375 melanoma cell lines; FIG. 3A, bottom panel—shows the densitometric quantitation of western blots. FIG. 3B shows the results of sandwich ELISA assays of biomarker IL-8 in melanoma cell line conditioned media with high (1205Lu, WM9, WM1361A, and WM1346) or low (WM793, WM1366, WM164, A375) basal autophagy. P-values were calculated using Welch's t-test (N=8; *p<0.05, **p<0.01). Error bars represent standard errors. Bars identifying cell lines are shown adjacent to FIG. 3F. FIG. 3C shows the results of sandwich ELISA assays of biomarker IL-1β as described in FIG. 3B. FIG. 3D shows the results of sandwich ELISA assays of biomarker LIF as described in FIG. 3B. FIG. 3E shows the results of sandwich ELISA assays of biomarker FAM3C as described in FIG. 3B. FIG. 3F shows the results of sandwich ELISA assays of biomarker DKK3 as described in FIG. 3B.

FIGS. 4A through 4F show the measurement of candidate biomarkers of high autophagy in Tat-Beclin 1 treated melanoma cells. FIG. 4A shows LC3 immunoblotting of WM793 cells treated with either Tat-Beclin 1, Tat-Scrambled, or no peptide in biological duplicates. FIG. 4B shows the results of a sandwich ELISA assay for LIF in conditioned medium of WM793 cells treated with either Tat-Beclin 1, Tat-Scrambled control, or no peptide. Error bars denote SD, and p-values were calculated by the Student's t-test (N=3 per treatment group) (*p<0.05, **p<0.01). FIG. 4C shows the results of a sandwich ELISA assay for IL-8 under the same conditions as FIG. 4B. FIG. 4D shows the results of a sandwich ELISA assay for IL-1β under the same conditions as FIG. 4B. FIG. 4E shows the results of a sandwich ELISA assay for FAM3C under the same conditions as FIG. 4B. FIG. 4F shows the results of a sandwich ELISA assay for DKK3 under the same conditions as FIG. 4B.

FIGS. 5A-5E show the measurement of candidate autophagy biomarkers in a high autophagy melanoma cell line with siRNA-induced Atg7 silencing. FIG. 5A shows levels of IL-8 measured by sandwich ELISA in the conditioned media of control- and mock-transfected WM1346 high autophagy melanoma cells vs. Atg7-silenced cells. Error bars denote SD, and p-values were calculated by ANOVA (*p<0.05, **p<0.01). FIG. 5B shows levels of IL-1β measured as described in FIG. 5A. FIG. 5C shows levels of LIF measured as described in FIG. 5A. FIG. 5D shows levels of DKK3 measured as described in FIG. 5A. FIG. 5E shows levels of FAM3C measured as described in FIG. 5A.

FIGS. 6A-6C demonstrate the measurement of autophagy candidate biomarkers in the serum of melanoma patients whose tumors have high or low autophagy. FIG. 6A shows representative electron micrographs (left panels) and vesicle counts from patients with low and high autophagy melanoma tumors (right panel). FIG. 6B shows the representative immunohistochemical staining of Atg5 in melanoma tumors from a patient with low and a patient with high tumor autophagy. FIG. 6C shows the measurement of IL-8, IL-1β, LIF, DKK3, and FAM3C by ELISA in serum of melanoma patients with either high or low levels of autophagy. Mean values are indicated by a horizontal line. P-values were calculated using Welch's t-test (N=5 per group).

FIG. 7 is a schematic showing workflow for the comparative secretome analysis of high-and low-autophagy cells. Schematic of the label-free approach used to identify differentially secreted proteins in the conditioned media (M) and the interstitial fluid (IF) of WM793 and 1205Lu Cells.

FIGS. 8A and 8B show the SDS-PAGE separation of melanoma conditioned medium and secretome reproducibility. FIG. 8A demonstrates “preparative” mini-gels of conditioned medium of low and high autophagy melanoma cell lines. Gels were run to 2-cm length and excised into twenty 1-mm fractions. FIG. 8B show the total protein and peptide identifications by cell culture compartment and by biological and technical replicates in 1205Lu and WM793 cell lines.

FIGS. 9A and 9B show the total protein overlap across biological and technical replicates. FIG. 9A shows the use of 1205Lu. FIG. 9B shows the use of WM793.

FIGS. 10A and 10B shows the statistical analysis of protein intensity variation across replicates. FIG. 10A shows the protein intensity comparison of technical replicates, with associated 95% confidence intervals for 1205Lu and WM793. FIG. 10B shows the protein intensity comparison of supernatant and interstitial fluid compartments by cell line with overlaid 95% confidence intervals.

FIG. 11 shows SAM analysis and data filtering. SAM plot illustrating observed scores plotted as a function of permuted/expected scores. False discovery rate was set based on the Δ tuning parameter. Data points in green represent proteins elevated in 1205Lu, whereas data points in red are those elevated in WM793 (FDR=3.52%).

FIG. 12 is a Western blot showing Atg7 silencing in WM1346 melanoma cell line. The blot shows Atg7 and LC3 expression in WM1346 cells 96 hours post-transfection with 20 nM siRNA.

### DETAILED DESCRIPTION OF THE INVENTION

Protein abundance levels of biomarkers in blood, in some embodiments, are dependent upon expression levels in tissues of origin (e.g., melanoma tumors), as well as rate of shedding into the blood and rate of clearance from the blood. While increased expression in a tumor often will correlate with increased abundance levels being observed in the blood, this is not necessarily always true. Therefore, the methods and compositions in one aspect refer to compositions that detect protein biomarkers and to protein assay methods. However, one of skill in the art, given the teachings contained herein, would readily understand that nucleic acid expression levels of the biomarkers and reagents and methods for their detections may be similarly practiced, without undue experimentation.

Certain secreted proteins were determined to be useful as biomarkers associated with tumor cell autophagy, e.g., in a model of melanoma. In one embodiment, these biomarkers included one or more of IL-8, IL-1β, DKK-3, FAM-3C and LIF. In other embodiments, these biomarkers further include one or more additional biomarkers selected from QPCT, SDCBP, APOD and ECM1. In still other embodiments, one or more additional biomarkers selected from Table 1 are also included.

As described in more detail in the Examples below, the secretome of low autophagy WM793 melanoma cells was compared to its highly autophagic metastatic derivative, 1205Lu, grown in physiological three dimensional cell culture using quantitative proteomics. These comparisons identified candidate autophagy biomarkers (IL-1β, IL-8, LIF, FAM3C, and DKK3) with known roles in inflammation and tumorigenesis and these proteins were subsequently shown to be elevated in supernatants of an independent panel of high autophagy melanoma cell lines. Secretion levels of these proteins increased when low autophagy melanoma cells were treated with an autophagy-inducing tat-Beclin 1 peptide³¹, and decreased when Atg7 was silenced in high autophagy cells, supporting a mechanistic link between these secreted proteins and intracellular autophagy. Further, serum collected from untreated metastatic melanoma patients with high tumor autophagy levels exhibited higher levels of these proteins than serum from patients with low autophagy tumors. These results demonstrate that autophagy-related secretion affects the tumor microenvironment and that measurement of extracellular autophagy-associated secreted proteins in tumors and in plasma can serve as surrogates for intracellular autophagy dynamics in tumor cells. Quantitative molecular profiling of the cell secretome can identify autophagy-related secretion of proteins that are promising vectors for monitoring intracellular autophagy dynamics in tumor cells. These proteins correlated with autophagy levels in an independent set of melanoma cell lines, confirming the proteomics results. Far more striking and interesting was the significant elevation of these candidate autophagy markers in the sera of patients with melanoma tumors with autophagy levels compared to patients with low tumor autophagy levels, given the innately higher level of complexity in tumor biology and secretion in vivo. While the initial patient sampling of the examples is small, these results strongly demonstrate development of a biomarker panel to monitor melanoma tumor autophagy levels. Furthermore, treatment of the low autophagy WM793 cell line with the targeted autophagy inducer Tat-Beclin 1 confirmed the autophagy dependence of the differential secretions observed in both the panel of cell lines and the clinical samples. Similar studies with an Atg7 silencing further supports the mechanistic link between these biomarkers and cell autophagy levels. These data are described in the examples below.

### I. DEFINITIONS

“Patient” or “subject” as used herein means a mammalian animal, including a human, a veterinary or farm animal, a domestic animal or pet, and animals normally used for clinical research. In one embodiment, the subject of these methods and compositions is a human.

The term “cancer” or “proliferative disease” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. The methods described herein are useful for the diagnosis and/or monitoring of cancer and tumor cells, i.e., both malignant and benign tumors, so long as the cells to be treated have an increase in autophagy levels as described herein. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, leukemia, myeloma, lymphoma, glioma, Non-Hodgkin's lymphoma, leukemia, multiple myeloma and multidrug resistant cancer. Whenever the term “melanoma” is used herein, it is used as a representative cancer for demonstration of the use of the methods and biomarker signatures and panels described herein.

By “biomarker” or “biomarker signature” as used herein is meant a single protein or a combination of proteins or peptide fragments thereof, the protein levels or relative protein levels or ratios of which significantly change (either in an increased or decreased manner) from the level or relative levels present in a subject having one physical condition or disease or disease stage from that of a reference standard representative of another physical condition or disease stage. Throughout this specification, wherever a particular biomarker is identified by name, it should be understood that the term “biomarker” includes IL-8, IL-1β, DKK-3, FAM2C, and LIF. These biomarkers may be combined to form certain sets of biomarkers or ligands to biomarkers in diagnostic reagents. Still other “additional” biomarkers are mentioned specifically herein in combination with one or more of IL-8, IL-1β, DKK-3, FAM2C, and LIF, include QPCT, SDCBP, APOD and/or ECM1, and optionally other biomarkers listed in Table 1 below. Biomarkers described in this specification include any physiological molecular forms, or modified physiological molecular forms, isoforms, pro-forms, and peptide fragments thereof, unless otherwise specified. It is understood that all molecular forms useful in this context are physiological, e.g., naturally occurring in the species. Preferably the peptide fragments obtained from the biomarkers are unique sequences. However, it is understood that other unique fragments may be obtained readily by one of skill in the art in view of the teachings provided herein.

By “isoform” or “multiple molecular form” is meant an alternative expression product or variant of a single gene in a given species, including forms generated by alternative splicing, single nucleotide polymorphisms, alternative promoter usage, alternative translation initiation small genetic differences between alleles of the same gene, and posttranslational modifications (PTMs) of these sequences.

By “related proteins” or “proteins of the same family” are meant expression products of different genes or related genes identified as belonging to a common family. Related proteins in the same biomarker family may or may not share related functions. Related proteins can be readily identified as having significant sequence identity either over the entire protein or a significant part of the protein that is typically referred to as a “domain”; typically proteins with at least 20% sequence homology or sequence identity can be readily identified as belonging to the same protein family.

By “homologous protein” is meant an alternative form of a related protein produced from a related gene having a percent sequence similarity or identity of greater than 20%, greater than 30%, greater than 40%, greater than 50%, greater than 60%, greater than 70%, greater than 75%, greater than 80%, greater than 85%, greater than 90%, greater than 95%, greater than 97%, or greater than 99%.

“Reference standard” as used herein refers to the source of the reference biomarker protein abundance, activity or encoding nucleotide sequence expression levels. The “reference standard” is preferably provided by using the same assay technique as is used for measurement of the subject's biomarker levels in the reference subject or population, to avoid any error in standardization. The reference standard is, alternatively, a numerical value, a predetermined cutpoint, a mean, an average, a numerical mean or range of numerical means, a numerical pattern, a ratio, a graphical pattern or a protein abundance profile or activity level profile derived from the same biomarker or biomarkers in a reference subject or reference population. In one embodiment, expression of nucleic acid sequences encoding the biomarkers forms the reference standard.

“Reference subject” or “Reference Population” defines the source of the reference standard. In one embodiment, the reference is a human subject or a population of subjects having no cancer or cells with elevated autophagy levels, i.e., healthy controls or negative controls. In yet another embodiment, the reference is a human subject or population of subjects with a cancer characterized by abnormally low or high autophagy levels. In yet another embodiment, the reference standard is a subject or population with one or more clinical indicators of melanoma. In still another embodiment, the reference is a human subject or a population of subjects having benign nodules or cysts. In still another embodiment, the reference is a human subject or a population of subjects who had melanoma, following surgical removal of a melanoma tumor. In another embodiment, the reference is a human subject or a population of subjects prior to surgical removal of an melanoma tumor. Similarly, in another embodiment, the reference is a human subject or a population of subjects following therapeutic treatment for melanoma, e.g., treatment with autophagy inhibitors. In still another embodiment, the reference is a human subject or a population of subjects prior to therapeutic treatment with autophagy inhibitors. In still other embodiments of methods described herein, the reference is obtained from the same test subject who provided a temporally earlier biological sample. That sample can be pre- or post-therapy or pre- or post-surgery.

Other potential reference standards are obtained from a reference that is a human subject or a population of subjects having early stage melanoma. In another embodiment the reference is a human subject or a population of subjects having advanced stage melanoma. In another embodiment, the reference standard is a combination of two or more of the above reference standards.

Selection of the particular class of reference standards, reference population, biomarker levels or profiles depends upon the use to which the diagnostic/monitoring methods and compositions are to be put by the physician and the desired result, e.g., initial diagnosis of a cancer, e.g., melanoma or other melanoma condition, clinical management of patients with cancer, e.g., melanoma, after initial diagnosis, including, but not limited to, monitoring for reoccurrence of disease or monitoring remission or progression of the cancer and either before, during or after therapeutic or surgical intervention, selecting among therapeutic protocols for individual patients (e.g., predicting the suitability of treatment with an autophagy inhibitor), monitoring for development of toxicity or other complications of therapy, predicting development of therapeutic resistance, and the like. Such reference standards or controls are the types that are commonly used in similar diagnostic assays for other biomarkers.

“Sample” as used herein means any biological fluid or tissue that contains the autophagy biomarkers identified herein. The most suitable samples for use in the methods and with the diagnostic compositions or reagents described herein are samples which require minimal invasion for testing, e.g., blood samples, including serum, plasma, whole blood, and circulating tumor cells. It is also anticipated that other biological fluids, such as saliva or urine, vaginal or cervical secretions, and ascites fluids or peritoneal fluid may be similarly evaluated by the methods described herein. Also, circulating tumor cells or fluids containing them are also suitable samples for evaluation in certain embodiments of this invention. The samples may include biopsy tissue, tumor tissue, surgical tissue, circulating tumor cells, or other tissue. Such samples may further be diluted with saline, buffer or a physiologically acceptable diluent. Alternatively, such samples are tested neat. In another embodiment, the samples are concentrated by conventional means. In certain embodiments, e.g., those in which expression levels of nucleic acid sequences encoding the biomarkers are desired to be evaluated, the samples may include biopsy tissue, surgical tissue, circulating tumor cells, or other tissue. In one embodiment, the sample is a tumor secretome, i.e., any fluid or medium containing the proteins secreted from the tumor. These shed proteins may be unassociated, associated with other biological molecules, or enclosed in a lipid membrane such as an exosome. In another embodiment, the sample is plasma.

By “significant change in biomarker level” is meant an increased protein level or activity of a selected biomarker in comparison to that of the selected reference standard or control or relative to a predetermined cutpoint; a decreased protein level of a selected biomarker in comparison to that of the selected reference or control or relative to a predetermined cutpoint; or a combination of a pattern or relative pattern of certain increased and/or decreased biomarkers. If appropriate, and in another embodiment, the biomarker levels refer to expression levels of the nucleic acid sequences encoding the biomarkers.

The degree of change in biomarker protein level can vary with each individual and is subject to variation with each population. For example, in one embodiment, a change of about a 1.5 or 5 or 10, 20 30, 40 or up to 50 fold increase or decrease, in protein levels of one or a small number of biomarkers, e.g., from 1, 2, 3, 4 or 5 of the characteristic biomarkers, is statistically significant. In another embodiment, statistically significant changes in biomarker protein level can include changes resulting in a “pattern” of changes in a panel of 5 to 10 or more biomarkers, including the autophagy biomarkers identified herein and additional biomarkers from Table 1. The degree of change in any biomarker(s) expression varies with the baseline, e.g., a previous measurement in the sample of the same patient or a baseline generated from a large number of samples of patients sharing the same condition, such as type of cancer, e.g., melanoma and with the size or spread of the cancer or solid tumor. The degree of change also varies with the immune response of the individual and is subject to variation with each individual. For example, in one embodiment of this invention, a change at or greater than a 1.5 fold increase or decrease in protein level of a biomarker or two such biomarkers, or even 3 or more biomarkers, is statistically significant. In another embodiment, a larger change, e.g., at or greater than a 2 fold increase or a decrease in expression of a biomarker(s) is statistically significant. This is particularly true for cancers without solid tumors. Still alternatively, if a single biomarker protein level is significantly increased in biological samples which normally do not contain measurable protein levels of the biomarker, such increase in a single biomarker level may alone be statistically significant. Conversely, if a single biomarker protein level is normally decreased or not significantly measurable in certain biological samples which normally do contain measurable protein levels of the biomarker, such decrease in protein level of a single biomarker may alone be statistically significant.

A change in protein level of a biomarker required for diagnosis or detection by the methods described herein refers to a biomarker whose protein level or activity is increased or decreased in a subject having a condition or suffering from a disease, e.g., melanoma, relative to its expression in a reference subject or reference standard. Biomarkers may also be increased or decreased in protein level at different stages of the same disease or condition. The protein levels of specific biomarkers differ between normal subjects and subjects suffering from a disease, benign melanoma nodules, or cancer, or between various stages of the same disease. Protein/activity levels of specific biomarkers differ between pre-surgery and post-surgery patients with melanoma. Such differences in biomarker levels include both quantitative, as well as qualitative, differences in the temporal or relative protein level or abundance patterns among, for example, biological samples of normal and diseased subjects, or among biological samples which have undergone different disease events or disease stages. For the purpose of this invention, a significant change in biomarker protein levels when compared to a reference standard is considered to be present when there is a statistically significant (p<0.05) difference in biomarker protein level between the subject and reference standard or profile, or significantly different relative to a predetermined cut-point.

For example, in one embodiment, the test subject's biomarker(s) levels are compared with a healthy reference standard. If the subject has melanoma, the selected autophagy biomarker(s), e.g., IL-8, IL-1β, DKK-3, FAM-3C or LIF, will typically show a change in protein level from the levels in the healthy reference standard, thus permitting diagnosis of melanoma. In another example, these biomarker(s) differentially change in protein level (either by increased or decreased protein level) when the biomarker levels or relative levels from the sample of a subject having one of the following conditions is compared to a reference subject or population having another of the following physical conditions. These “conditions” include no melanoma, the presence of benign melanoma nodules, the presence of an melanoma or subtype, the condition following surgical removal of an melanoma tumor; the condition prior to surgical removal of an melanoma tumor; the condition following a specific therapeutic treatment for an melanoma tumor; the condition prior to a specific therapeutic treatment for an melanoma tumor. It is further anticipated that the biomarker(s) expression levels may change and the changes may be detected during treatment for melanoma. Still other embodiments of “conditions” as defined above include early stage melanoma; advanced stage melanoma; or chemo-resistant melanoma.

In another example, a change in the selected autophagy biomarkers, e.g., IL-8, IL-1β, DKK-3, FAM-3C or LIF, and/or QPCT, SDCBP, APOD and ECM1, and/or additional biomarkers from Table 1, can permit a diagnosis of another cancer, e.g., pancreatic cancer or a host of other cancers which have tumors that shed or secrete these biomarkers in to the tissue or bloodstream as the disease progresses.

The term “ligand” with regard to protein biomarkers refers to a molecule that binds or complexes, with a biomarker protein, molecular form or peptide, such as an antibody, antibody mimic or equivalent, a molecular form or fragment thereof. In certain embodiments, in which the biomarker expression is to be evaluated, the ligand can be a nucleotide sequence, e.g., polynucleotide or oligonucleotide, primer or probe.

As used herein, the term “antibody” refers to an intact immunoglobulin having two light and two heavy chains or fragments thereof capable of binding to a biomarker protein or a fragment of a biomarker protein. Thus a single isolated antibody or fragment may be a monoclonal antibody, a synthetic antibody, a recombinant antibody, a chimeric antibody, a humanized antibody, a human antibody, or a bio-specific antibody or multi-specific construct that can bind two or more target biomarkers. The term “antibody fragment” refers to less than an intact antibody structure, including, without limitation, an isolated single antibody chain, an Fv construct, a Fab construct, an Fc construct, a light chain variable or complementarity determining region (CDR) sequence, etc.

As used herein, “labels” or “reporter molecules” are chemical or biochemical moieties useful for labeling a ligand, e.g., amino acid, peptide sequence, protein, or antibody. “Labels” and “reporter molecules” include fluorescent agents, chemiluminescent agents, chromogenic agents, quenching agents, radionucleotides, enzymes, substrates, cofactors, inhibitors, radioactive isotopes, magnetic particles, and other moieties known in the art. “Labels” or “reporter molecules” are capable of generating a measurable signal and may be covalently or noncovalently joined to a ligand.

As used herein the term “cancer” refers to or describes the physiological condition in mammals that is typically characterized by unregulated cell growth. More specifically, as used herein, the term “cancer” means any melanoma, particularly those with high autophagy levels. The term “tumor,” as used herein, refers to all neoplastic cell growth and proliferation, whether malignant or benign, and all pre-cancerous and cancerous cells and tissues.

By “therapeutic reagent” or “regimen” is meant any type of treatment employed in the treatment of cancers with or without solid tumors, including, without limitation, chemotherapeutic pharmaceuticals, biological response modifiers, radiation, diet, vitamin therapy, hormone therapies, gene therapy, surgical resection, etc. In one particular embodiment, a therapeutic regimen includes the administration of an autophagy inhibitor such as hydroxychloroquine.

The term “microarray” refers to an ordered arrangement of binding/complexing array elements or ligands, e.g. antibodies, on a substrate.

In the context of the compositions and methods described herein, reference to “at least two,” “at least five,” etc. of the autophagy biomarkers in any particular biomarker set means any and all combinations of the biomarkers identified above or in combination with biomarkers of Table 1. Specific biomarkers for the biomarker profile do for use in this invention include any of the biomarker sets identified as (a) to (y) below, but may also include any biomarker, fragment or molecular form.

By “significant change in expression” is meant an upregulation in the expression level of a nucleic acid sequence, e.g., genes or transcript, encoding a selected biomarker, in comparison to the selected reference standard or control; a downregulation in the expression level of a nucleic acid sequence, e.g., genes or transcript, encoding a selected biomarker, in comparison to the selected reference standard or control; or a combination of a pattern or relative pattern of certain upregulated and/or down regulated biomarker genes. The degree of change in biomarker expression can vary with each individual as stated above for protein biomarkers.

The term “polynucleotide,” when used in singular or plural form, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The term “polynucleotide” specifically includes cDNAs. The term includes DNAs (including cDNAs) and RNAs that contain one or more modified bases. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide of less than 20 bases, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

One skilled in the art may readily reproduce the compositions and methods described herein by use of the amino acid sequences of the biomarkers and other molecular forms, which are publicly available from conventional sources.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also be described using “consisting of” or “consisting essentially of” language.

The term “a” or “an”, refers to one or more, for example, “a biomarker,” is understood to represent one or more biomarkers. As such, the terms “a” (or “an”), “one or more,” and “at least one” are used interchangeably herein.

As used herein, the term “about” means a variability of 10% from the reference given, unless otherwise specified.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### II. BIOMARKERS AND LIGAND SETS USEFUL IN THE METHODS AND COMPOSITIONS

The “target biomarkers” of the compositions and methods of these inventions include, in one aspect, IL-1β, IL-8, LIF, DKK-3 and FAM3C, optionally with other biomarkers QPCT, SDCBP, APOD and/or ECM1, as well as others identified in Table 1, fragments, particularly unique fragments thereof, and molecular forms thereof. Differences in basal autophagy in human melanoma manifest as secretory changes in novel serological biomarkers of autophagy for cancer. In one embodiment, these identified target autophagy biomarkers are IL-1β, IL-8, LIF, FAM3C, and DKK3, which have known roles in inflammation and tumorigenesis. Inflammatory cytokines are a particularly important subclass of these autophagy biomarkers. Network analysis indicated strong functional linkages of inflammatory proteins with motility and growth-promotion, emphasizing the importance of cytokine regulation networks in the tumor microenvironment. Furthermore, secretion of oncogenic cytokines has been shown to directly induce autophagy²⁰. IL-1β, IL-8, LIF, and FAM3C are all cytokines that regulate immune and inflammatory processes, representing 80% of the validated biomarkers.

In one embodiment, these biomarkers may alone provide information regarding the autophagy levels of a biological composition. In another embodiment, taken together, this panel of biomarkers provides a unique window into how intracellular autophagy can control extracellular processes that impact the tumor microenvironment and increase the malignant potential of cancer cells. The examples below demonstrate the feasibility of utilizing these biomarkers in a clinical setting. These candidate biomarkers that performed well in cell line and patient serum validation sets are anticipated to be useful in a multiplexed plasma protein based assay and will serve as a prognostic, predictive, or pharmacodynamic biomarker panel to aid cancer therapy decisions in melanoma. These biomarkers are also targets for developing non-invasive clinically-applicable methods of monitoring autophagy in cancer cells.

These proteins were subsequently shown to be elevated in supernatants of an independent panel of high autophagy melanoma cell lines. Secretion levels of these proteins increased when low autophagy melanoma cells were treated with an autophagy-inducing tat-Beclin 1 peptide³¹, supporting a potential mechanistic link between these secreted proteins and intracellular autophagy. Further, serum collected from untreated metastatic melanoma patients with high tumor autophagy levels exhibited higher levels of these proteins than serum from patients with low autophagy tumors. These results show that autophagy-related secretion affects the tumor microenvironment and that measurement of extracellular autophagy-associated secreted proteins in tumors and in plasma can serve as surrogates for intracellular autophagy dynamics in tumor cells.

Diagnostic reagents that can detect and measure these target autophagy biomarkers and sets of biomarkers identified herein and methods for evaluating the level or ratios of these target biomarkers vs. their level(s) in a variety of reference standards or controls of different conditions or stages in various cancers, including melanoma, are valuable tools in the early detection and monitoring of melanoma.

In certain embodiments, superior diagnostic tests for diagnosing the existence of melanoma utilize at least one of the ligands that bind or complex with one or more of the five autophagy biomarkers, or one of the fragments or molecular forms thereof. In other embodiments, superior diagnostic tests for distinguishing melanoma from one of the conditions recited above utilize multiple ligands, each individually detecting a different specific target biomarker identified herein, or isoform, modified form or peptide thereof. In still other methods, no ligand is necessary, e.g., MRM assays.

Thus, in one aspect, the target biomarker of the methods and compositions described herein is interleukin 1 beta (IL-1β, or an isoform, pro-form, modified molecular form, or unique peptide fragment or nucleic acid fragment thereof. IL-1β is a cytokine that promotes inflammation, angiogenesis and tissue remodeling³⁴. Cleaved by caspase-1 to an active form by an NALP3 inflammasome complex, IL-1β lacks a signal peptide and is released to the extracellular matrix via autophagosomes¹¹. While the interplay of autophagy and inflammasome activation remain controversial, dysregulated inflammasome activity can promote carcinogenic inflammation via IL-1β secretion³⁵⁻³⁷. In particular, late-stage melanoma cells secrete IL-1β constitutively via the inflammasome, promoting angiogenesis and invasiveness³⁴. The amino acid sequences for IL-1β and its molecular forms are publically available, such as in GENBANK. In one embodiment, the biomarker is UniProtKB/Swiss-Prot: P01584.2, or a peptide fragment thereof. Certain fragments of IL-β that may be useful as targets in the methods and compositions described herein include one or more unique peptide fragments. It should be understood that, depending upon the context, any reference to IL-1β herein also refers to a peptide and the molecular form thereof, as well as the nucleotide sequences encoding IL-1β and/or any of its unique peptides or forms.

In another aspect, the target biomarker of the methods and compositions described herein is interleukin 8 (IL-8), or an isoform, pro-form, modified molecular form, or unique peptide fragment or nucleic acid fragment thereof. IL-8 is a chemokine produced by macrophages and epithelial cells, inducing chemotaxis in target cells and promoting angiogenesis³⁸. IL-8 is known to be secreted by melanoma cells and correlates with metastatic potential by up-regulating matrix metalloproteinase 2, a candidate biomarker identified in our study and secreted at higher levels in 1205Lu cells³⁹. IL-8 secretion increases tumorigenicity and cell proliferation. The amino acid sequences for IL-8 and its molecular forms are publically available, such as in GENBANK. In one embodiment, the biomarker is NCBI Reference Sequence: NP_{—}000575.1 or a peptide fragment thereof. Certain fragments of IL-8 that may be useful as targets in the methods and compositions described herein include one or more unique peptide fragments. It should be understood that, depending upon the context, any reference to IL-8 herein also refers to a peptide and the molecular form thereof, as well as the nucleotide sequences encoding IL-8 and/or any of its unique peptides or forms.

In another aspect, the target biomarker of the methods and compositions described herein is Leukemia Inhibitory Factor (LIF) or an isoform, pro-form, modified molecular form, or unique peptide fragment or nucleic acid fragment thereof. LIF is a cytokine involved in a number of processes: hematopoietic differentiation, stem cell development, bone metabolism, and growth promotion⁴⁰. LIF-producing melanoma is known to result in bone metastasis and subsequent osteclastogenesis, leading to bone tissue degradation⁴¹. Through the activation of JAK-STAT and MAPK signaling, LIF induces the expression of IL-8 and IL-1β; hence, there remains the possibility that LIF up-regulates IL-8 and IL-1β for their coordinated secretion through autolysosomes to promote melanoma progression and invasiveness. The amino acid sequences for LIF and its molecular forms are publically available, such as in GENBANK. In one embodiment, the biomarker is UniProtKB/Swiss-Prot: P15018.1 or a peptide fragment thereof. Certain fragments of LIF that may be useful as targets in the methods and compositions described herein include one or more unique peptide fragments. It should be understood that, depending upon the context, any reference to LIF herein also refers to a peptide and the molecular form thereof, as well as the nucleotide sequences encoding LIF and/or any of its unique peptides or forms.

In another aspect, the target biomarker of the methods and compositions described herein is FAM3C or an isoform, pro-form, modified molecular form, or unique peptide fragment or nucleic acid fragment thereof. FAM3C, also known as interleukin related protein (ILEI), is a member of a group of secreted proteins with largely unknown function. FAM3C has been found to be both necessary and sufficient for epithelial to mesenchymal transition, tumor formation, and metastasis^{30,42}. Through the up-regulation of cytokines, chemokines, growth factor receptors, and cell signaling intermediates, FAM3C can trigger an autocrine growth factor and chemokine loop that is particularly relevant to cancer progression. The robust elevations of these cytokines in melanoma cell conditioned media and patient sera suggest that high basal autophagy plays a critical role in the progression and aggressiveness of malignant melanoma via inflammatory cytokines. The amino acid sequences for FAM3C and its molecular forms are publically available, such as in GENBANK. In one embodiment, the biomarker is NCBI Reference Sequence: NP_{—}001035109.1 or a peptide fragment thereof. Certain fragments of FAM3C that may be useful as targets in the methods and compositions described herein include one or more unique peptide fragments. It should be understood that, depending upon the context, any reference to FAM3C herein also refers to a peptide and the molecular form thereof, as well as the nucleotide sequences encoding FAM3C and/or any of its unique peptides or forms.

In another aspect, the target biomarker of the methods and compositions described herein is dickkopf protein family 3 (DKK3) or an isoform, pro-form, modified molecular form, or unique peptide fragment or nucleic acid fragment thereof. DKK3, a cysteine-rich, N-glycosylated secreted protein of the dickkopf protein family, is the remaining high priority biomarker in this study. DKK3 does not have a clearly defined relationship to cytokine activity or inflammation, but it is implicated in key tumor progression processes. DKK glycoproteins are temporally and spatially regulated to influence cell fate decisions and the epithelial to mesenchymal transition⁴³⁻⁴⁷. DKK3 has also been found to play a role in tumor angiogenesis, through its differentiating effect on tumor-associated endothelial cells⁴⁷. Further, higher expression levels of DKK3 have been reported in melanoma, suggesting that secretion of DKK3 in aggressive, high autophagy melanomas may support in vivo angiogenesis and invasiveness. The amino acid sequences for DKK3 and its molecular forms are publically available, such as in GENBANK. In one embodiment, the biomarker is GenBank: AAQ88744.1 or a peptide fragment thereof. Certain fragments of DKK3 that may be useful as targets in the methods and compositions described herein include one or more unique peptide fragments. It should be understood that, depending upon the context, any reference to DKK3 herein also refers to a peptide and the molecular form thereof, as well as the nucleotide sequences encoding DKK3 and/or any of its unique peptides or forms.

In still another embodiment, the target biomarker is actually a set of targets. A biomarker combination includes, without limitation, or consists of, the following exemplary combinations of biomarkers for diagnosis of high autophagy cancers, e.g., melanoma, or for monitoring the progression of the severity of disease or remission of disease:
(a) IL-8 and IL-1β,
(b) IL-8 and DKK-3,
(c) IL-8 and FAM-3C
(d) IL-8 and LIF
(e) IL-8, IL-1β and DKK-3,
(f) IL-8, IL-1β, and FAM-3C,
(g) IL-8, IL-1β, and LIF
(h) IL-8, DKK-3 and FAM-3C
(i) IL-8, DKK-3 and LIF
(j) IL-8, FAM-3C and LIF
(k) IL-8, DKK-3, FAM-3C and LIF
(l) IL-8, IL-1β, FAM-3C and LIF
(m) IL-8, IL-1β, DKK-3, and FAM-3C
(n) IL-8, IL-1β, DKK-3, and LIF
(o) IL-8, IL-1β, DKK-3, FAM-3C and LIF
(p) IL-1β and DKK-3,
(q) IL-1β and FAM-3C
(r) IL-1β and LIF
(s) IL-1β, DKK-3, and FAM-3C
(t) IL-1β, LIF and FAM-3C,
(u) IL-1β, DKK-3 and LIF
(v) IL-1β, DKK-3, FAM-3C and LIF
(w) DKK-3 and FAM-3C
(x) DKK-3 and LIF, or
(y) DKK-3, LIF, and FAM-3C.

However, still other additional biomarkers that can be added to any of sets (a) through (y) can include QPCT, SDCBP, APOD and/or ECM1, and/or any of the biomarkers identified in Table 1 below. One of skill in the art can readily combine various sets of these biomarkers to form panels in addition to the exemplary panels (a) through (y) above.

For example, among desirable biomarker signatures are signatures that comprise, or consist of, at least 2, 3, 4, 5, 6, 7, 8, 9, 10, 15, 20, 25 or more of the biomarkers described herein or any other known melanoma biomarker or molecular forms or peptides thereof

As further stated above, the biomarkers/biomarker signatures described above, can in another embodiment, refer to nucleic acid sequences, genes and transcripts encoding the biomarkers and expression profiles thereof

### III. DIAGNOSTIC REAGENTS, DEVICES AND KITS

Thus, in one aspect, a diagnostic reagent, kit or device comprises two or more biomarkers selected from:
(a) IL-8,
(b) IL-1f3,
(c) DKK-3,
(d) FAM-3C,
(e) LIF,
(f) one or more additional biomarkers QPCT, SDCBP, APOD and/or ECM1;
(g) one or more additional biomarkers of Table 1;
(i) an isoform, pro-form, modified molecular form, posttranslational modification, or unique peptide fragment or unique nucleic acid fragment of any of (a) through (g), or
(h) a protein in the same biomarker family or expressed from a related gene having at least 20% sequence homology or sequence identity with any biomarker (a) to g).

In another aspect, a diagnostic reagent, kit or device comprises two or more ligands selected from:
(a) a ligand capable of specifically complexing with, binding to, or quantitatively detecting or identifying IL-8,
(b) a ligand capable of specifically complexing with, binding to, or quantitatively detecting or identifying IL-1β,
(c) a ligand capable of specifically complexing with, binding to, or quantitatively detecting or identifying DKK-3,
(d) a ligand capable of specifically complexing with, binding to, or quantitatively detecting or identifying FAM-3C,
(e) a ligand capable of specifically complexing with, binding to, or quantitatively detecting or identifying LIF,
(f) a ligand capable of specifically complexing with, binding to, or quantitatively detecting or identifying one or more additional biomarkers QPCT, SDCBP, APOD and/or ECM1;
(g) a ligand capable of specifically complexing with, binding to, or quantitatively detecting or identifying one or more additional biomarkers one or more additional biomarkers of Table 1;
(h) a ligand capable of specifically complexing with, binding to, or quantitatively detecting or identifying an isoform, pro-form, modified molecular form, posttranslational modification, or unique peptide fragment or unique nucleic acid fragment of any of (a) through (g), or
(i) a ligand capable of specifically complexing with, binding to, or quantitatively detecting or identifying a protein in the same biomarker family or expressed from a related gene having at least 20% sequence homology or sequence identity with any biomarker (a) to (g).

In one embodiment, the biomarker protein or nucleic acid or ligand thereto is associated with a molecule or moiety capable alone or in combination with one or more additional molecules of generating a detectable signal. In another embodiment, the biomarker protein or nucleic acid or ligand is associated with a substrate and is immobilized.

In certain aspects, the diagnostic reagent or device includes ligands which can include an antibody or fragment of an antibody, an antibody mimic, a synthetic antibody, a single chain antibody, a bi-specific antibody or multi-specific constructs, or an equivalent that binds to or complexes with a single biomarker or to multiple of the biomarkers, said ligand optionally associated with a detectable label or with a substrate. Such antibodies may be presently extant in the art or presently used commercially, such as those available as part of commercial antibody sandwich ELISA assay kits or that may be developed by techniques now common in the field of immunology. A recombinant molecule bearing the binding portion of a biomarker antibody, e.g., carrying one or more variable chain CDR sequences that bind e.g., FAM-3C, etc. may also be used in a diagnostic assay. As used herein, the term “antibody” may also refer, where appropriate, to a mixture of different antibodies or antibody fragments that bind to the selected biomarker. Such different antibodies may bind to different biomarkers or different portions of the same biomarker protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the compositions or methods described herein may be generated synthetically or recombinantly, using conventional techniques or may be isolated and purified from plasma or further manipulated to increase the binding affinity thereof. It should be understood that any antibody, antibody fragment, or mixture thereof that binds one of the biomarkers described herein or a particular sequence of the selected biomarker or peptide fragment thereof may be employed in the compositions and methods, regardless of how the antibody or mixture of antibodies was generated. Various forms of antibody, e.g., polyclonal, monoclonal, recombinant, chimeric, as well as fragments and components (e.g., CDRs, single chain variable regions, etc.) or antibody mimics or equivalents may be used in place of antibodies. The ligand itself may be labeled or immobilized.

In another embodiment, the reagent ligands are nucleotide sequences, the diagnostic reagent is a polynucleotide or oligonucleotide sequence that hybridizes to gene, gene fragment, gene transcript or nucleotide sequence encoding a biomarker discussed herein or encoding a unique peptide thereof. Such a polynucleotide/oligonucleotide can be a probe or primer, and may itself be labeled or immobilized. In one embodiment, ligand-hybridizing polynucleotide or oligonucleotide reagent(s) are part of a primer-probe set, and the kit comprises both primer and probe. Each said primer-probe set amplifies a different gene, gene fragment or gene expression product that encodes a different biomarker discussed herein, optionally including one or more additional known biomarkers, also as described above. For use in the compositions the PCR primers and probes are preferably designed based upon intron sequences present in the biomarker gene(s) to be amplified selected from the gene expression profile. The design of the primer and probe sequences is within the skill of the art once the particular gene target is selected. The particular methods selected for the primer and probe design and the particular primer and probe sequences are not limiting features of these compositions. A ready explanation of primer and probe design techniques available to those of skill in the art is summarized in U.S. Pat. No. 7,081,340, with reference to publically available tools such as DNA BLAST software, the Repeat Masker program (Baylor College of Medicine), Primer Express (Applied Biosystems); MGB assay-by-design (Applied Biosystems); Primer3 (Steve Rozen and Helen J. Skaletsky (2000) Primer3 on the WWW for general users and for biologist programmers and other publications.

In general, optimal PCR primers and probes used in the compositions described herein are generally 17-30 bases in length, and contain about 20-80%, such as, for example, about 50-60% G+C bases. Melting temperatures of between 50 and 80° C., e.g. about 50 to 70° C. are typically preferred.

In one embodiment, such a ligand binding to a protein biomarker or a unique peptide contained therein, can be an antibody which specifically binds a single biomarker such as, or a unique peptide in that single biomarker or a nucleic acid sequence which hybridizes to the nucleic acid sequence encoding or its unique peptide. In one embodiment, such a ligand desirably binds to a protein biomarker or a unique peptide contained therein, and can be an antibody which specifically binds to one or more of the biomarker family members, individually, collectively, or individually to a unique peptide in that single biomarker.

Thus, a diagnostic reagent, kit or device as described herein can comprise 1 to 2, 3, 4, 5, 6, 7, 8, 10, 15 or 20 or more biomarker sequences or ligands thereto (including those identified in Table 1). In another embodiment, suitable labeled or immobilized reagents include at least 2, 3, 4, 5, 6, 7 8, 9, 10 or 11 or more ligands or antibodies, in which each ligand binds to or complexes with a single biomarker protein/peptide, fragment, or molecular form of the biomarker(s) listed in detail above.

In another embodiment, a diagnostic reagent or device comprises biomarkers or multiple ligands directed to two, three, four or five of the IL-8, IL-1β, DKK-3, FAM-3C or LIF autophagy biomarkers, or one or more additional biomarkers QPCT, SDCBP, APOD and/or ECM1 or markers from Table 1, a common or shared sequence formed of two or more of these related proteins, or multiple isoforms, pro-forms, modified molecular forms, or unique peptide fragments or nucleic acid fragments thereof. In one embodiment, the biomarker proteins or nucleic acid sequences or ligands thereto are each associated with a molecule or moiety capable alone or in combination with one or more additional molecules of generating a detectable signal. In another embodiment, the biomarker protein or nucleic acid or ligands thereto is associated with a substrate and is immobilized. Where the reagent or device contains ligands to multiple biomarker sequences, or multiple biomarker ligands, the detectable moieties and signals can each be different, so as to identify different results.

In such embodiments of the diagnostic reagents, kits and devices, at least one of the biomarkers or ligands is associated with a detectable label or portion of a detectable label system. In another embodiment, a diagnostic reagent includes one or more target biomarker or peptide fragment identified herein or ligand thereto, immobilized on a substrate. In still another embodiment, combinations of such labeled or immobilized biomarkers or ligands are suitable reagents and components of a diagnostic kit or device. In another aspect, suitable embodiments of such labeled or immobilized reagents include at least one, 2, 3, 4, 5, 6, 7, 8, 9, 10 or all 20 or more biomarkers identified herein or their unique peptide fragments or ligands thereto.

Any combination of labeled or immobilized biomarkers or labeled or immobilized ligands thereto as described above can be assembled in a diagnostic kit or device for the purposes of diagnosing cancer cells demonstrating high autophagy levels, including melanoma, such as those combinations of biomarkers discussed herein.

For these reagents, the labels may be selected from among many known diagnostic labels, including those described above. Similarly, the substrates for immobilization in a device may be any of the common substrates, glass, plastic, a microarray, a microfluidics card, a glass slide, a chip, a bead or a chamber.

Any combination of labeled or immobilized biomarker ligands can be assembled in a diagnostic kit or device for the purposes of diagnosing melanoma.

Thus, a kit or device can contain multiple biomarker targets as reagents or one or more ligands to each biomarker target. For example, one embodiment of a composition includes a substrate upon which the biomarkers or ligands are immobilized. In another embodiment, the kit also contains optional detectable labels, immobilization substrates, optional substrates for enzymatic labels, as well as other laboratory items.

The diagnostic reagents, devices, or kits compositions based on the biomarkers or fragments described herein or including the ligands thereto, optionally associated with detectable labels, can be presented in the format of a microfluidics card, a chip or chamber, a bead or a kit adapted for use with assays formats such as sandwich ELISAs, multiple protein assays, platform multiplex ELISAs, such as the BioRad Luminex platform, mass spectrometry quantitative assays, or PCR, RT-PCR or Q PCR techniques.

The selection of the ligands, biomarker sequences, their length, suitable labels and substrates used in the reagents and kits are routine determinations made by one of skill in the art in view of the teachings herein of which biomarkers form signatures suitable for the diagnosis of melanoma. Assembly of the ligands and biomarkers discussed herein, attachment to a substrate, and assembly into the form of a microarray, a microfluidics card, a chip, a bead, or a chamber employ techniques known in the art.

### IV. METHODS FOR DIAGNOSING OR MONITORING CANCERS WITH HIGH AUTOPHAGY LEVELS

In another embodiment, a method for diagnosing or detecting or monitoring the progress of melanoma and treatment of melanoma in a subject comprises, or consists of, a variety of steps.

In another embodiment, a method for identifying the autophagy level of a cell in a biological sample of a subject comprises, or consists of, a variety of steps. The method involves contacting a biological sample containing cells obtained from a test subject with a diagnostic reagent or device or kit as described above. From this sample, or from a protein level profile generated from the sample, the protein/activity level of at least one autophagy biomarker is measured or identified. A significant modulation in protein level of an autophagy biomarker in the subject's sample relative to a reference standard is determined. The modulation correlates with an increase or decrease in the autophagy level in the subject's cells.

In another embodiment an assay method for predicting the sensitivity of a subject with cancer to treatment with an autophagy inhibitor comprises or consists of several steps. A biological sample obtained from a test subject is contacted with a diagnostic reagent or device as described above. The protein levels/activity of at least one autophagy biomarker is detected or measured in the sample or from a protein level profile generated from the sample. The protein level of the biomarker in the subject's sample is evaluated relative to the level of the same biomarker in a temporally earlier sample from the subject, a subject's sample prior to treatment with the inhibitor, or a reference standard from healthy subjects. A significant increase in biomarker level in the subject's sample relative to that in the reference standard indicates the subject's response to the inhibitor. This method can indicate whether a particular subject's cancer is suitable for treatment with an autophagy inhibitor.

In another embodiment, an assay method for assessing the chemo-resistance of a cancer comprises or consists of several steps. A biological sample obtained from a test subject is contacted with a diagnostic reagent or device or kit as described herein; and the protein levels/activity level of one of more of the autophagy biomarkers or sets described herein is detected or measure in the sample or from a protein level profile generated from the sample. The protein level of the biomarker in the subject's sample is evaluated relative to the level of the same biomarker in a temporally earlier sample from the subject, either treated or untreated with a chemotherapeutic, a reference standard from subjects with cancer that is not chemoresistant, or a reference standard from healthy subjects. A significant increase in biomarker level in the subject's sample relative to that in the reference standards indicates the subject's cancer is chemoresistant.

In yet another embodiment, a method for treating a subject with melanoma comprises or consists of several steps. A biological sample obtained from a test subject is contacted with a diagnostic reagent or device or kit as described herein. Tumor autophagy levels in the subject are quantitatively measured by detecting or measuring in the sample or from a protein level profile generated from the sample, the protein levels of at least one autophagy biomarker or biomarker set. The protein level/activity of the biomarkers in the subject's sample are evaluated relative to the level of the same biomarker in a healthy reference standard or in a reference standard of subjects with non-chemoresistant melanoma. Thereafter, the subject is treated with an autophagy inhibitor, when the biomarker expression levels in the subject's tumor sample are increased over that of the reference standard.

According to any of these embodiments, the subject may have a cancer or tumor, such as melanoma. In one embodiment, in all methods, the sample is obtained by minimally invasive means. In certain aspects, the sample is preferably blood, plasma or serum. In another embodiment, a suitable sample is urine. In other embodiments, the sample can be biopsy tissue. The reference standard is a mean, an average, a numerical mean or range of numerical means, a numerical pattern, a ratio, a graphical pattern or a protein level profile derived from the same biomarker or biomarkers in a reference subject or reference population.

In certain of these methods, when the change or modulation is an increase in expression of the biomarker level relative to a healthy reference standard, the sample contains cancer cells. In other aspects of these methods, when the modulation is a decrease in expression of the biomarker level relative to a reference standard characteristic of a subject with an aggressive or chemo-resistant cancer, the sample does not contain chemoresistant cancer cells or contains cancer cells responding positively to cancer treatment.

The steps of these methods are described in more detail below.

A. Sample Preparation

The test sample is obtained from a human subject who is to undergo diagnosis, selection of treatment or is in the process of being treated. The subject's sample can in one embodiment be provided before initial diagnosis, so that the method is performed to diagnose the existence of a melanoma, e.g., one with high autophagy levels. In another embodiment, depending upon the reference standard and markers used, the method is performed to diagnosis the stage of melanoma. In another embodiment, the subject's sample can be provided after a diagnosis, so that the method is performed to monitor progression of the cancer. In another embodiment, the sample can be provided prior to surgical removal of a melanoma tumor or prior to therapeutic treatment of a diagnosed melanoma and the method used to thereafter monitor the effect of the treatment or surgery, and to check for relapse. In another embodiment, the sample can be provided following surgical removal of a melanoma tumor or following therapeutic treatment of a diagnosed melanoma, and the method performed to ascertain efficacy of treatment or relapse. In yet another embodiment the sample may be obtained from the subject periodically during therapeutic treatment for a melanoma, and the method employed to track efficacy of therapy or relapse. In yet another embodiment the sample may be obtained from the subject periodically during therapeutic treatment to enable the physician to change therapies or adjust dosages. In one or more of these embodiments, the subject's own prior sample can be employed in the method as the reference standard. Finally, the methods can be employed before or after the subject receives an autophagy inhibitor to assess the need for, or efficacy of the inhibitor.

Preferably where the sample is a fluid, e.g., blood, serum or plasma, obtaining the sample involves simply withdrawing and preparing the sample in traditional fashion for contact with the diagnostic reagent. Where the sample is a tissue or tumor sample, it may be prepared in conventional manner for contact with the diagnostic reagent.

The method further involves contacting the sample obtained from a test subject with a diagnostic reagent as described above under conditions that permit the reagent to bind to or complex with one or more biomarker(s) and/or additional biomarkers which may be present in the sample. This method may employ any of the suitable diagnostic reagents or kits or compositions described above.

B. Measuring Biomarker Levels

Thereafter, a suitable assay is employed to detect or measure in the sample the protein level (actual or relative) of one or more biomarker(s), including and/or multiple biomarkers or biomarker sets, and/or one or more additional biomarkers discussed herein in Table 1. Alternatively, a suitable assay is employed to generate a protein abundance profile (actual or relative or ratios thereof) of multiple biomarkers discussed herein from the sample.

In another embodiment, the above method further includes measuring in the biological sample of the subject the protein levels of two or more additional biomarkers which form with one and/or multiple other biomarkers of Table 1, a biomarker protein signature for high autophagy levels. In one embodiment, the measurement of all target biomarkers occurs in a single sample. In another embodiment, the measurement of all target biomarkers occurs in a multiple samples from a single patient. It should be understood that the measurement of all biomarkers need not occur simultaneously or in the same assay. Results from multiple assays may be combined providing that they are performed within a reasonable time for comparison with the other target biomarker levels.

The measurement of the biomarker(s) in the biological sample may employ any suitable ligand, e.g., antibody, antibody mimic or equivalent (or antibody to any second biomarker) to detect the biomarker protein, as described above. Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method for a single biomarker, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods and devices of this invention are detectable by other means, e.g., colored latex microparticles (Bangs Laboratories, Indiana) in which a dye is embedded may be used in place of enzymes to provide a visual signal indicative of the presence of the resulting selected biomarker-antibody complex in applicable assays. Still other labels include fluorescent compounds, radioactive compounds or elements. Preferably, an anti-biomarker antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC+PE, FITC+PECy5 and PE+PECy7, among others may be used depending upon assay method.

Detectable labels for attachment to antibodies useful in diagnostic assays and devices of this invention may be easily selected from among numerous compositions known and readily available to one skilled in the art of diagnostic assays. The biomarker-antibodies or fragments useful in this invention are not limited by the particular detectable label or label system employed. Thus, selection and/or generation of suitable biomarker antibodies with optional labels for use in this invention is within the skill of the art, provided with this specification, the documents incorporated herein, and the conventional teachings of immunology.

Similarly the particular assay format used to measure the selected biomarker in a biological sample may be selected from among a wide range of protein assays, such as described in the examples below. Suitable assays include enzyme-linked immunoassays, sandwich immunoassays, homogeneous assays, immunohistochemistry formats, or other conventional assay formats. In one embodiment, a serum/plasma sandwich ELISA is employed in the method. In another embodiment, a mass spectrometry-based assay is employed. In another embodiment, a MRM assay is employed, in which antibodies are used to enrich the biomarker in a manner analogous to the capture antibody in sandwich ELISAs. One of skill in the art may readily select from any number of conventional immunoassay formats to perform this invention.

Other reagents for the detection of protein in biological samples, such as peptide mimetics, synthetic chemical compounds capable of detecting the selected biomarker may be used in other assay formats for the quantitative detection of biomarker protein in biological samples, such as high pressure liquid chromatography (HPLC), immunohistochemistry, etc.

Employing ligand binding to the biomarker proteins or multiple biomarkers forming the autophagy signature enables more precise quantitative assays, as illustrated by the multiple reaction monitoring (MRM) mass spectrometry (MS) assays. As an alternative to specific peptide-based MRM-MS assays that can distinguish specific protein isoforms and proteolytic fragments, the knowledge of specific molecular forms of biomarkers allows more accurate antibody-based assays, such as sandwich ELISA assays or their equivalent. Frequently, the isoform specificity, protein domain specificity and affects of posttranslational modifications on binding of immune reagents used in pre-clinical (and some clinical) diagnostic tests are not well defined.

In one embodiment, suitable assays for use in these methods include immunoassays using antibodies or ligands to the above-identified biomarkers and biomarker signatures. In another embodiment, a suitable assay includes a multiplexed MRM based assay for two more biomarkers that include one or more of the proteins/unique peptides described herein. It is anticipated that ultimately the platform most likely to be used in clinical assays will be multi-plexed or parallel sandwich ELISA assays or their equivalent, primarily because this platform is the technology most commonly used to quantify blood proteins in clinical laboratories. MRM MS assays may continue to be used productively to help evaluate the isoform/molecular form specificity of any existing immunoassays or those developed in the future. In addition, multiplexed quantitative MS assays such as MRM MS may replace ELISA assays in clinical laboratories in some situations.

C. Detection of a Change in Biomarker Level, Autophagy Signature and Diagnosis

The protein level of the one or more biomarker(s) in the subject's sample or the protein abundance profile or signature of multiple said biomarkers as detected by the use of the assays described above is then compared with the level of the same biomarker or biomarkers in a reference standard or reference profile. In one embodiment, the comparing step of the method is performed by a computer processor or computer-programmed instrument that generates numerical or graphical data useful in the appropriate diagnosis of the condition. Optionally, the comparison may be performed manually.

The detection or observation of a change in the protein level of a biomarker or biomarkers in the subject's sample from the same biomarker or biomarkers in the reference standard can indicate an appropriate diagnosis. An appropriate diagnosis can be identifying a risk of developing chemo-resistant melanoma, a diagnosis of melanoma (or stage or type thereof), a diagnosis or detection of the status of progression or remission of melanoma in the subject following therapy or surgery, a determination of the need for a change in therapy or dosage of therapeutic agent, including the susceptibility of the cancer to an autophagy inhibitor. The method is thus useful for early diagnosis of disease, for monitoring response or relapse after initial diagnosis and treatment or to predict clinical outcome or determine the best clinical treatment for the subject.

In one embodiment, the change in protein level of each biomarker can involve an increase of a biomarker or multiple biomarkers in comparison to the specific reference standard. In one embodiment, the biomarker is increased in a subject sample from a patient having melanoma when compared to the levels of these biomarkers from a healthy reference standard. In another embodiment, the biomarkers are increased in a subject sample from a patient having melanoma prior to therapy or surgery, when compared to the levels of these biomarkers from a post-surgery or post-therapy reference standard.

In another embodiment, the change in protein level of each biomarker can involve a decrease of a biomarker or multiple biomarkers in comparison to the specific reference standard. In one embodiment, the biomarkers are decreased in a subject sample from a patient having melanoma following surgical removal of a tumor or following chemotherapy/radiation when compared to the levels of these biomarkers from a pre-surgery/pre-therapy melanoma reference standard or a reference standard which is a sample obtained from the same subject pre-surgery or pre-therapy.

In still other embodiments, the changes in protein levels of the biomarkers may be altered in characteristic ways if the reference standard is a particular stage of melanoma, or if the reference standard is derived from benign melanoma cysts or nodules.

The results of the methods and use of the compositions described herein may be used in conjunction with clinical risk factors to help physicians make more accurate decisions about how to manage patients with melanomas. Another advantage of these methods and compositions is that diagnosis may occur earlier than with more invasive diagnostic measures.

D. Alternative Assay Embodiments

In an alternative embodiment, the method of diagnosis or risk of diagnosis involves using the nucleic acid hybridizing reagent ligands described above to detect a significant change in expression level of the subject's sample biomarker or biomarkers from that in a reference standard or reference expression profile which indicates a diagnosis, risk, or the status of progression or remission of melanoma in the subject. These methods may be performed in other biological samples, e.g., biopsy tissue samples, tissue removed by surgery, or tumor cell samples, including circulating tumor cells isolated from the blood, to detect or analyze a risk of developing a melanoma, as well as a diagnosis of same. Such methods are also known in the art and include contacting a sample obtained from a test subject with a diagnostic reagent comprising a ligand which is a nucleotide sequence capable of hybridizing to a nucleic acid sequence encoding a biomarker or biomarker combination described herein, e.g., FAM3C and IL-8 and/or multiple other autophagy proteins (see sets (a) through (y) above), said ligand associated with a detectable label or with a substrate. Thereafter one would detect or measure in the sample or from an expression profile generated from the sample, the expression levels of one or more of the biomarkers or ratios thereof. The expression level(s) of the biomarker(s) in the subject's sample or from an expression profile or ratio of multiple said biomarkers are then compared with the expression level of the same biomarker or biomarkers in a reference standard. A significant change in expression level of the subject's sample biomarker or biomarkers from that in the reference standard indicates a diagnosis, risk, or the status of progression or remission of melanoma in the subject.

Suitable assay methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

The comparison of the quantitative or relative expression levels of the biomarkers may be done analogously to that described above for the comparison of protein levels of biomarkers.

E. Non-Ligand-Based Analysis

In another aspect, the methods described above may be modified by using non-ligand based methods, such as mass spectrometry. For example, proteins in a biological sample obtained from a test subject may be contacted with a chemical or enzymatic agent and the proteins, including the biomarkers contained therein fragmented in the sample. The digested sample or portions thereof are injected into a mass spectrometer and the protein levels or ratios of one or more of the biomarker or biomarker combinations described herein, optionally with other known biomarkers, modified molecular forms, peptides and unique peptides or ratios thereof, are quantitatively identified or measured by mass spectrometry. The protein levels of the biomarkers in the subject's sample are then compared with the level of the same biomarker or biomarkers in a reference standard or to a predetermined cutoff derived from the reference standard. In one embodiment, the agent is a proteolytic enzyme. In another embodiment, the agent is trypsin.

A significant change in protein level of the subject's sample biomarker or biomarkers from that in the reference standard or from a predetermined cutoff indicates a diagnosis, risk, or the status of progression, chemoresistance or remission of melanoma in the subject.

Thus, the various methods, devices and steps described above can be utilized in an initial diagnosis of melanoma or other melanoma condition, as well as in clinical management of patients with melanoma after initial diagnosis. Uses in clinical management of the various devices, reagents and assay methods, include without limitation, monitoring for reoccurrence of disease or monitoring remission or progression of the cancer and either before, during or after therapeutic or surgical intervention, selecting among therapeutic protocols for individual patients, monitoring for development of toxicity or other complications of therapy, and predicting development of therapeutic resistance.

In one embodiment, the method involves enriching the biomarker protein or one or more peptides produced by specific proteolysis in the sample by contacting the sample with an antibody prior to injecting into a mass spectrometer in a manner analogous to a capture antibody in a conventional sandwich ELISA. In another embodiment, the method involves depleting the sample of non-target proteins prior to injecting sample into a mass spectrometer. The depletion may also be performed using antibodies to the non-targets. The method described herein may use liquid chromatographic mass spectrometry, such as HPLC. One such method is described in detail in the Examples below.

Use of the methods and reagents, kits or devices described herein permits early diagnosis of disease, monitoring relapse after initial diagnosis and treatment, predicting clinical outcome, or determining the best clinical treatment. These uses are particularly valuable for cancers which demonstrate high autophagy levels, such as melanoma.

### V. EXAMPLES

The invention is now described with reference to the following examples.

The molecular changes in the melanoma cell secretome were investigated as a means of identifying key proteins diagnostic of autophagy levels. To determine whether extracellular secreted proteins could serve as novel molecular markers of intracellular autophagy dynamics in tumor cells, basal autophagy levels of genetically paired melanoma cell lines were measured in physiological 3D cell culture using electron microscopy and LC3 immunoblotting.

To approximate autophagy dynamics in the tumor microenvironment, human melanoma cells were grown as spheroids in Type I bovine collagen matrices. To enrich for autophagy-related secretions, secretomes of low-autophagy WM793 cells and the genetically closely related high-autophagy, metastatic 1205Lu cells were directly compared at baseline. Cells were grown in physiological three dimensional cell culture and secretomes were compared using quantitative proteomics. Specifically, conditioned media was collected and separated using 1-D gel electrophoresis, digested with trypsin, and analyzed using liquid chromatography-tandem mass spectrometry analysis on an LTQ-Orbitrap hybrid instrument. Mass spectra were analyzed using the Rosetta ELUCIDATOR proteomic pipeline, and significance analysis of microarrays (SAM) was used to identify differentially secreted proteins. From Gene Ontology (GO) analysis, candidate biomarkers were enriched for extracellular matrix components, inflammatory mediators, and metabolic enzymes implicated in autophagy and melanoma progression. Network analysis using the shortest paths algorithm identified densely connected sub-networks that illustrated interplay between inflammatory cytokines and metastasis-related proteins, providing insight into molecular pathways that regulate constitutively elevated autophagy.

Briefly described, the conditioned media of these high and low autophagy melanoma cell lines were compared using label-free LC-MS/MS, and both biological and technical replicates were evaluated in order to determine reproducibility of the proteomics method. Fold-change, involvement in biological networks, and availability of high throughput assays were used as primary criteria to select five proteins as candidate biomarkers for further evaluation.

A panel of additional melanoma cell lines was assessed for basal autophagy through LC3 immunoblotting. Secreted levels of the top biomarker candidates were measured in conditioned media of three high and three low autophagy melanoma cell lines using sandwich ELISA assays. In addition, the low autophagy WM793 melanoma cell line was treated with tat-Beclin1 to induce higher autophagy levels. LC3 immunoblotting was used to confirm the autophagy-inducing effects of tat-Beclin1, and sandwich ELISA assays of conditioned medium were used to measure the effects of the tat-Beclin 1 treatment on secretion of the candidate biomarkers. Further, the high autophagy cell line, WM1346, was treated with siRNA duplexes targeting ATG7. LC3 and ATG7 immunoblotting were used to confirm the autophagy-modifying and knockdown effects, respectively. Sandwich ELISA assays of conditioned medium were used to measure the effects of ATG7 silencing on the secretion of the candidate biomarkers versus relevant controls, which include the parental cell line, the non-targeting siRNA treatment, and the treatment with transfection reagent alone.

To determine whether these candidate biomarkers correlated with tumor autophagy in plasma from patients, autophagy levels in tumor tissue derived from melanoma patients enrolled in a phase II trial were first confirmed by ATG5 immunohistochemistry and electron microscopy of tumor biopsies. Sera were collected prior to treatment with alkylating chemotherapy. Levels of top biomarker candidates were measured by sandwich ELISA in patient serum.

These examples are provided for the purpose of illustration only and the invention should in no way be construed as being limited to these examples but rather should be construed to encompass any and all variations that become evident as a result of the teaching provided herein.

### Example 1

### Materials and Methods

A. Cell Culture, Electron Microscopy, and Western Blot

Melanoma cell lines WM973 and 1205Lu (as well as other cell lines identified in the appropriate figures) were maintained in RPMI 1640 (Invitrogen) supplemented with 10% fetal bovine serum (Sigma), 50 μg/mL gentamicin, and 25 mmol/L HEPES in the presence of 5% CO₂ at 37° C. The 3D spheroid cultures were prepared as previously described¹ and cell death was evaluated using the Live/Dead Assay (Invitrogen). Electron microscopy was performed as previously described¹. Immunoblotting was performed on whole cell lysates as previously described. A commercially available polyclonal antibody against actin (I-19) was used (Santa Cruz). A polyclonal antibody against LC3 that was produced by QCB Biologicals was used. Band densities from Western Blots were quantified using Adobe Photoshop CS4 Extended.

B. Conditioned Media Collection

For optimal depth of secretome protein identification, cell culture medium was changed to 1% fetal calf serum and cells were incubated at 37° C. for 24 hours immediately prior to collection of the conditioned media. The conditioned medium of each cell line was collected, centrifuged, passed through a 0.22 μm filter, and concentrated 10-fold on a 10 kDa MWCO concentration unit (Millipore).

C. SDS-PAGE, In-Gel Digestion, and Nano LC-MS/MS

Concentrated conditioned media were separated on a 12% NuPage mini-gel with MES running buffer until the tracking dye migrated 20 mm and the gel was stained using colloidal Coomassie (Invitrogen, Carlsbad, Calif.). In-gel digestion and LC-MS/MS was performed as described previously⁴⁸ using a FASTA database (human UniRef 100, Ver. May, 2011) downloaded from Protein Information Resource (PIR), Georgetown University, Washington, D.C. Raw files were imported and analyzed for label-free quantitation in the Rosetta Elucidator software suite, as previously described⁴⁹. A minimum protein intensity cut-off of 1×10⁴ was used to minimize noise from weak mass spectrometry signals as these are typically variable. Derivation of 95% confidence intervals for technical replicate data was performed as previously described^{49,50}. Briefly, to define significance thresholds, data were normalized using the median of log 10 intensity ratio from technical replicate comparisons, and the intensity dependence of protein variation across technical replicates in each cell line was determined using a sliding window approach with protein intensities pooled into groups of 15. The standard deviation trend as a function of the mean intensity was fitted to an exponential curve and the 95% confidence boundaries for each cell line were derived from this curve, with the assumption that each protein follows a log-normal distribution⁴⁹. These boundaries were then overlaid on protein intensity scatterplots comparing the interstitial fluid versus the media, whereby outlying proteins represented those which preferentially reside to a given cell culture compartment.

D. Bioinformatics Analysis

Only proteins identified with at least two unique peptides were used. Signal intensities of unique peptides were used for protein quantification. For comparison of media and interstitial fluid and combined protein levels, the initial values obtained from the proteome analysis were corrected for the proportion of the initial volumes of media and interstitial fluid recovered and used for analysis. Significance Analysis of Microarrays (SAM) was used to select differentially secreted proteins across cell lines, as described previously¹⁵. SAM was implemented in RExcel using a two-class unpaired d-statistic, 200 permutations, and a k-nearest neighbor imputer for missing values. The false discovery rate (<5%) was estimated as the ratio of false positives (FP) to true positives (TP) among proteins called significant using the Δ tuning parameter.

The Software Tool for Rapid Annotation of Proteins (http://www.bumc.bu.edu/cardiovascularproteomics/cpctools/strap/) was used to classify proteins by molecular and biological function and by subcellular localization based on Gene Ontology annotation. Differentially secreted proteins were analyzed for biological pathways through an integrative analysis using Biological Networks version 2.0 (University of California San Diego, San Diego, Calif.). Biological Networks is a software suite that enables integrative analysis of interaction networks, signaling pathways, gene regulatory modules, and genomic sequences^{51,52}. Uniprot accession IDs were uploaded into Biological Networks for database searching and network analysis using the shortest paths algorithm, with minimum probabilities for STRING functional relations (co-expression and co-citation) set to 0.9 (high stringency). Betweenness-centrality clustering was used to identify subnetworks, with a minimum and maximum number of nodes per cluster set to 10 and 20, respectively, and a minimum p-value of 0.01.

E. Quantitation of Selected Candidate Biomarkers of Autophagy in Conditioned Media

ELISA kits for IL-8, IL-1β, LIF from Life Technologies, FAM3C from MyBioSource, and DKK3 from Sigma were used to quantitate the target proteins in conditioned media using specimens described below.

Basal autophagy phenotype was evaluated in a panel of melanoma cell lines by LC3 immunoblotting (Low—WM1366, WM164, A375; High—WM9, WM1361A, WM1346). 1205Lu and WM793 were included to compare results to those obtained in the proteomics analysis. Cell lines in exponential growth phase (seeded at 2×10⁵/well in 1 mL) were grown in RPMI-1640 supplemented with 8% fetal calf serum Immunoblotting was performed on whole cell lysates using actin (I-19) (Santa Cruz), and the polyclonal antibody against LC3 from QCB Biologicals.

Tat-Beclin 1 peptide (735 μM stock solution in 1×PBS) was diluted in Opti-MEM (Life Technologies) acidified with 0.15% 6N HCl, as previously described³¹. WM793 cells (2×10⁵/well in 1 mL) adapted in RPMI 1640/1% FCS were incubated for either 3 h or 6 h in 10 μM Tat-Beclin 1 or Tat-Scrambled media and 3 h only in 20 μM Tat-Beclin 1 or Tat-Scrambled. Cells incubated for 3 h in Opti-HCl with or without Tat-Beclin 1 or Tat-Scrambled control peptide were returned to RPMI-1640 supplemented with 1% FCS for 3 hours. Conditioned medium was collected for further analysis, and cells were lysed in 1% SDS/20 mM Tris (pH 8) for western blot analysis. Immunoblotting was performed on whole cell lysates using actin (I-19) (Santa Cruz), and a polyclonal antibody against LC3 from QCB Biologicals.

F. Transfections with siRNA 439

WM1346 human melanoma cell lines in exponential growth phase were plated at 2×10⁵ 440 cells/well in 1 mL of media for 24 hours before transfection. Cells were then transfected with either 20 nM siRNA targeting Atg7 transcripts, 20 nM non-targeting control siRNA (HS Negative Control siRNA, Qiagen), 20 nM positive control siRNA (HS Cell Death Positive Control, Qiagen), or transfection reagent alone. Three Atg7 siRNA duplexes were purchased from Qiagen: siRNA-1, 5′-CAGCATCATCTTCGAAGTGAA-3′; siRNA-2, 5′-CAAGAGAAAGCTGGTCATCAA-3′; siRNA-3, 5′-ATCAGTGGATCTAAATCTCAA-3′. The siRNAs were dissolved in RNase-free water provided by the manufacturer to prepare stock solutions. Transfection complexes were prepared by mixing appropriate volumes of Opti-MEM 448 with siRNA solution and transfection reagent (Lipofectamine RNAiMAX, Life Technologies). Transfection complexes were incubated at 37° C. for 16 hours, after which cells were returned to normal growth medium. After 72 hours, growth medium was replaced and conditioned medium was collected after 24 hours. Conditioned medium was probed by sandwich ELISA as described above.

G. Patient Samples

In all cases informed consent on an IRB-approved tissue collection protocol was obtained prior to biopsy or retrieval of archival tumor tissue. In this study, archived sera from patients enrolled on a phase II trial of temozolomide and sorafenib in advanced melanoma were used (53). Both tumor tissue and serum were collected pretreatment. ATG5 immunohistochemistry was conducted using Novus Biologics antibody (NB110-53818). Sandwich ELISA assays, described above, were used to quantitate target proteins in serum.

H. Statistical Analysis

Welch's t-test was used to compare secretion in high vs. low autophagy melanoma cell lines and patient serum. (GraphPad Software, San Diego, Calif.). All comparisons were performed using a two-tailed test and p-values of <0.05 were considered significant.

### Example 2

### Determination of Autophagic Capacity in Metastatic 1205Lu and its Parental Cell Line, WM793

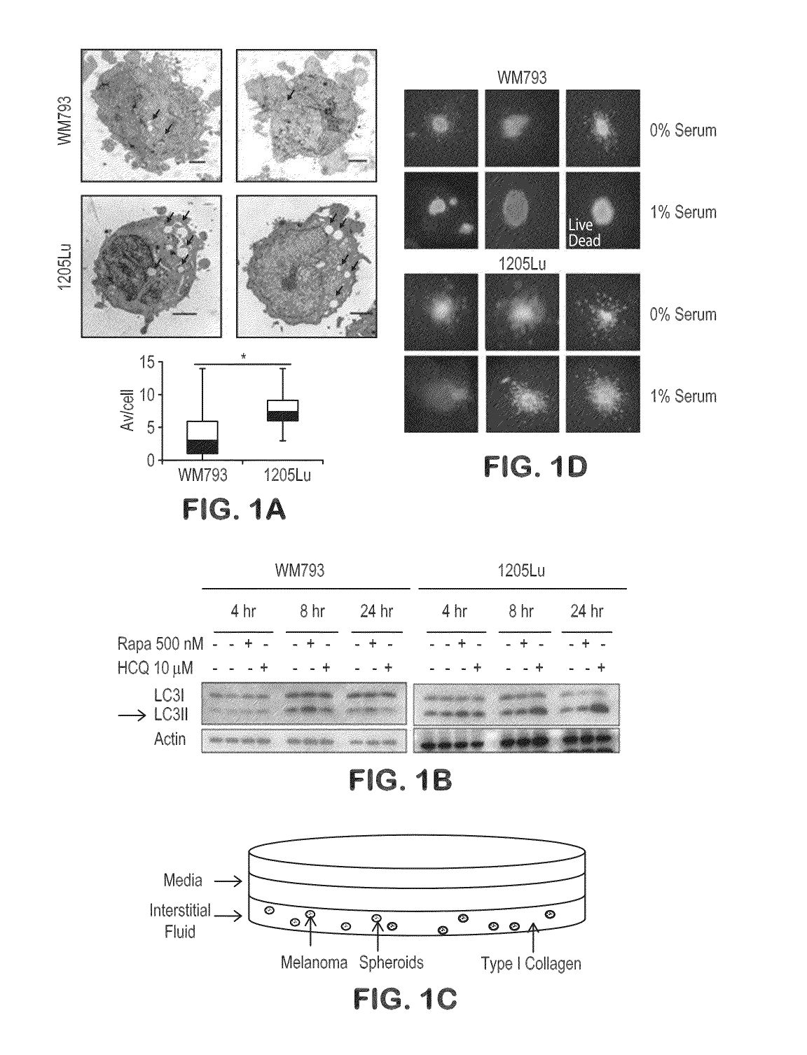

The genetically paired melanoma cell lines, WM793 and 1205Lu, were used for initial secretome comparisons due to the inherent genetic heterogeneity across unrelated melanoma cell lines. The 1205Lu cell line is a subclone derived from the lung metastasis of WM793 in athymic mice, thus minimizing protein expression changes caused by genetic differences¹⁴. To determine autophagic capacity in these paired cell lines, electron microscopy of WM793 and 1205Lu grown in complete medium was used to quantify AVs (FIG. 1A). The median number of AVs/cell in 1205Lu and WM793 cells was 8 compared to 4 (p=0.0003), respectively Immunoblot analysis of LC3 showed elevated PE-modified LC3 (LC3II) versus unmodified LC3-I for untreated 1205Lu cells compared with WM793 cells grown in complete medium. Further, modulation of autophagy with the autophagy inducer rapamycin, and a distal autophagy inhibitor, hydroxychloroquine, produced increases in the LC3II/LC3I ratio in 1205Lu cells compared to WM793 cells (FIG. 1B).

These results indicate that 1205Lu cells have increased autophagic capacity compared to WM793 cells. To better recapitulate the autophagy levels found in vivo, cells were grown as spheroids in Type I bovine collagen in either 0% or 1% bovine serum (FIG. 1C). Although serum-free conditions would enhance analytical depth of the secretome analysis using proteomics by avoiding interference from abundant serum proteins, substantial cell death was observed in the WM793 melanoma spheroids after 24 hours in culture with 0% bovine serum (FIG. 1D). No significant cell death was observed for either cell line when 3D spheroids were grown in 1% serum. Therefore, this condition was used for subsequent experiments.

### Example 3

### Evaluation of Proteins in the Interstitium and Media of Melanoma Cells in 3D Culture

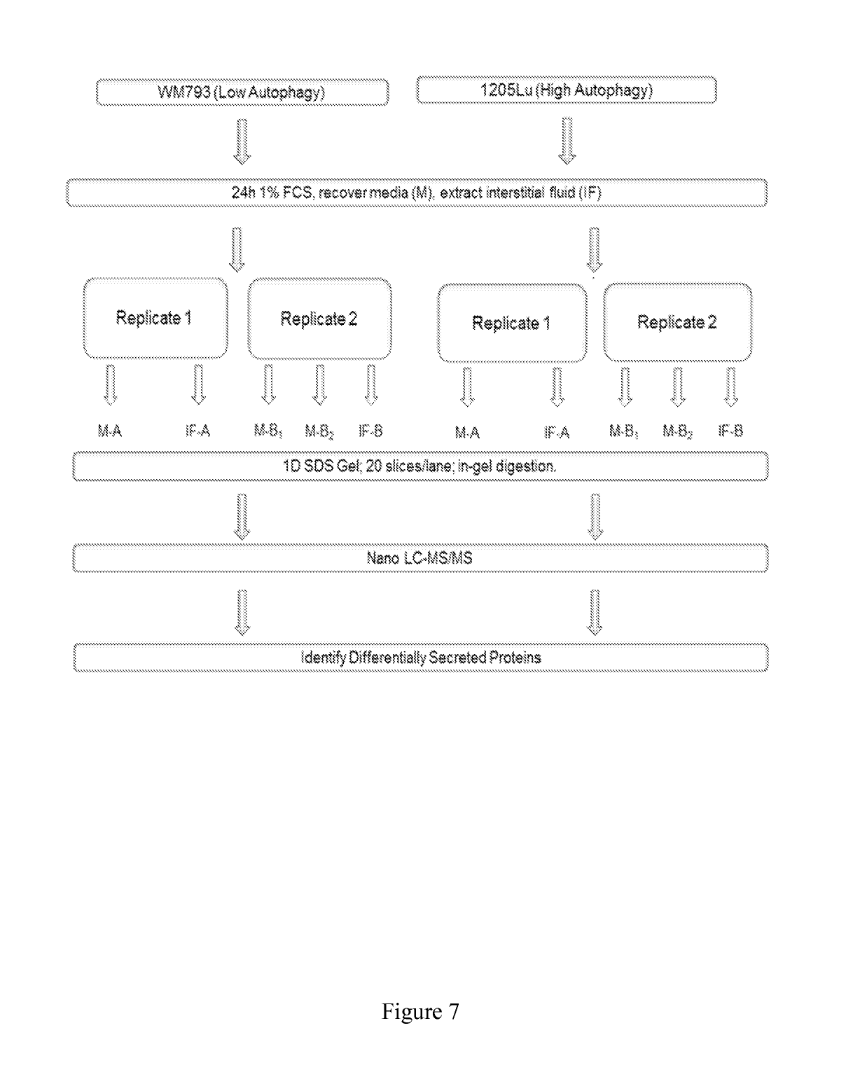

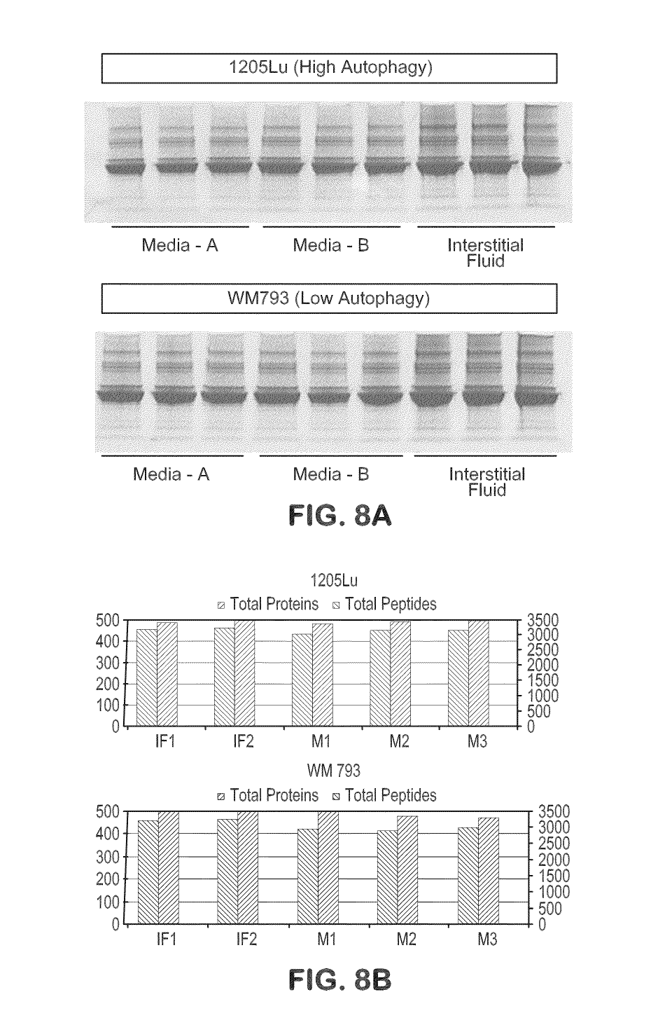

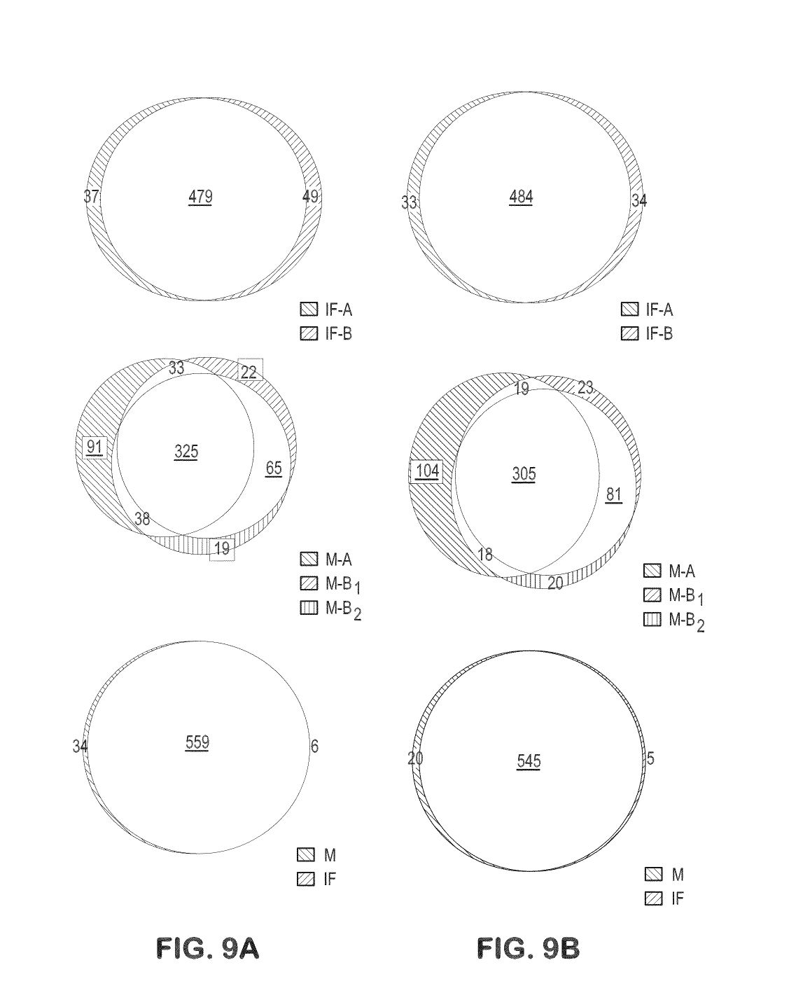

Conditioned medium from above the surface of the collagen (media) and conditioned media from the interstitial space (interstitial fluid) within the collagen matrix (FIG. 1C) were analyzed separately by GeLC-MS/MS (in-gel digestion followed by liquid chromatography and tandem mass spectrometry) as shown in FIG. 7. As illustrated, to determine the reproducibility of this proteome analysis method, 50-100 spheroids for each cell line were grown in collagen in duplicate wells to produce biological replicate samples. In addition, the media fractions from Replicate 2 were divided in half and processed separately as technical replicates. Each sample was separated on a short SDS gel and gel lanes were divided into 20 equal slices to improve depth of analysis (FIG. 8A). The total numbers of protein and peptide identifications across all replicates were very similar (FIG. 8B). The specific proteins identified in biological replicates for interstitial fluid as well as biological and technical replicates for media were very similar for each cell line (FIG. 9).

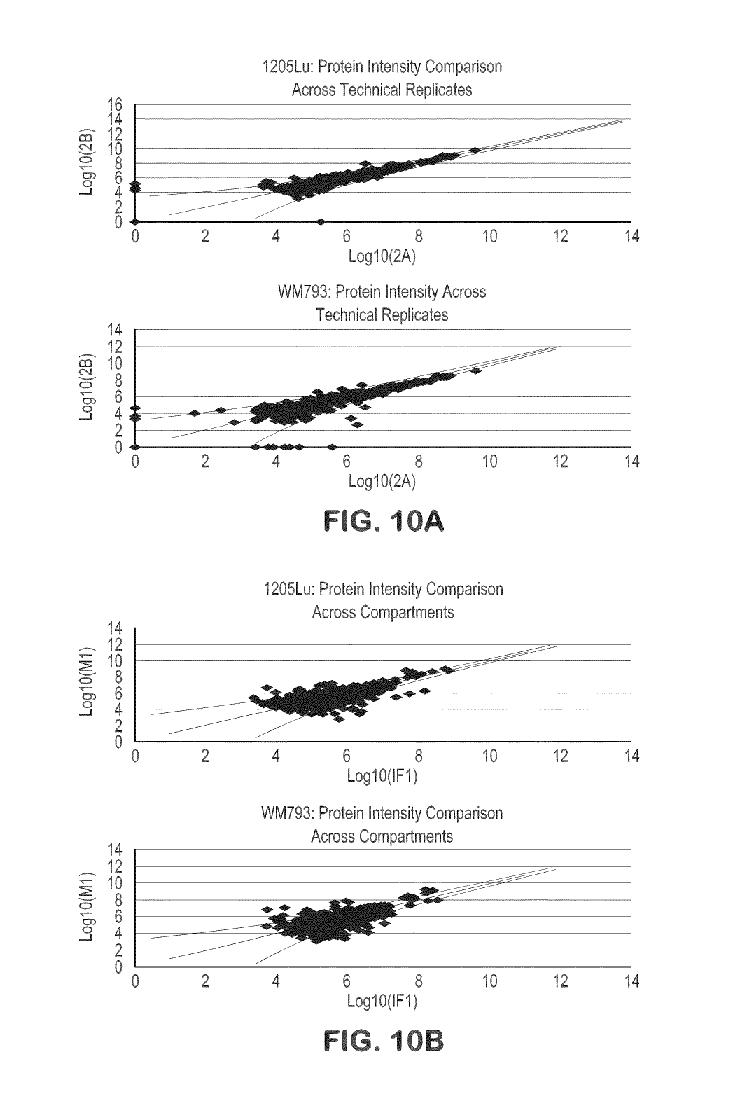

An important consideration when analyzing conditioned medium from spheroid cell cultures is the potential for selected proteins to preferentially remain associated with the collagen matrix rather than freely diffusing into the overlaying media layer. However, at the protein identification level only minor differences were observed between these two fractions (FIG. 9). To quantitatively evaluate potential systematic sequestration in the collagen matrix, log-transformed and normalized protein intensities were compared between the media and interstitial fluid fractions for each cell line using a 95% confidence level as described herein (FIG. 10). Analysis of media and interstitial fluid across all replicates for each cell line revealed that no protein or protein group reproducibly localized in the interstitial fluid. Hence, to avoid potentially random variability in partial distribution of a subset of proteins between fractions, protein intensities from the interstitial fluid and media data were combined in silico.

### Example 4

### Identification of Differentially Secreted Proteins Across 1205Lu and WM793 3D Spheroids

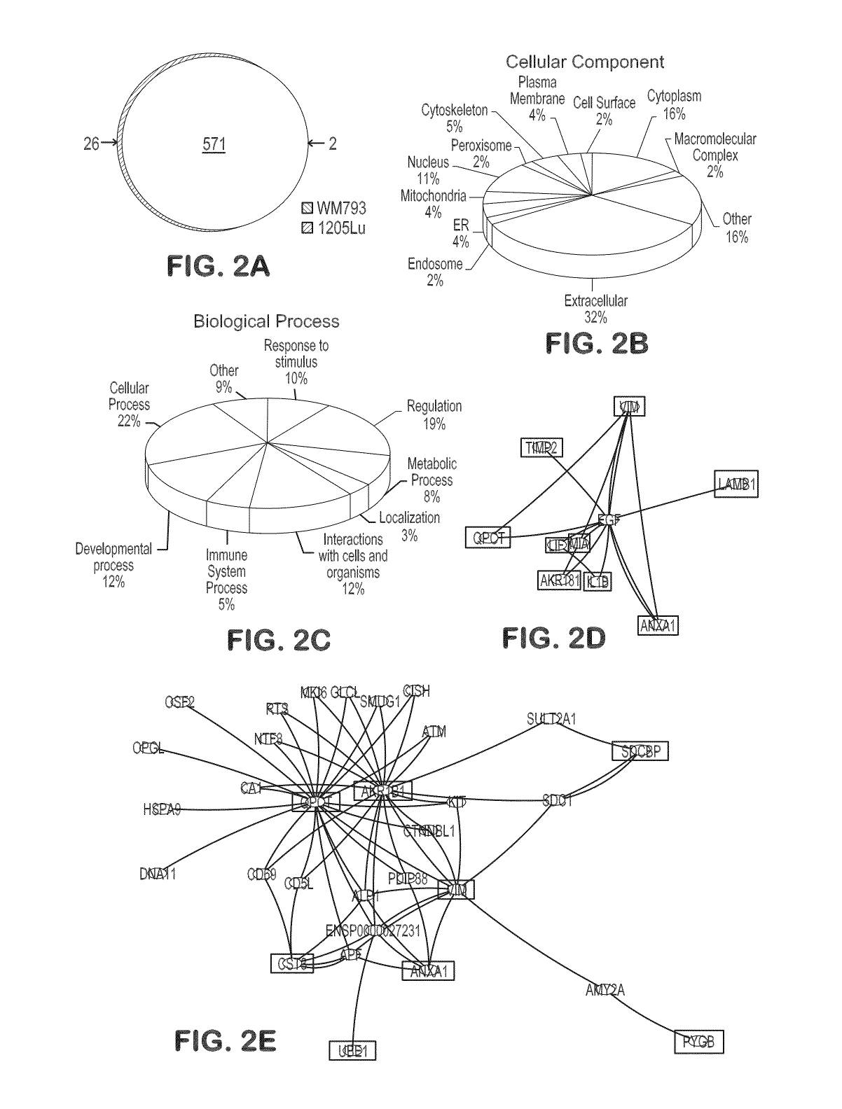

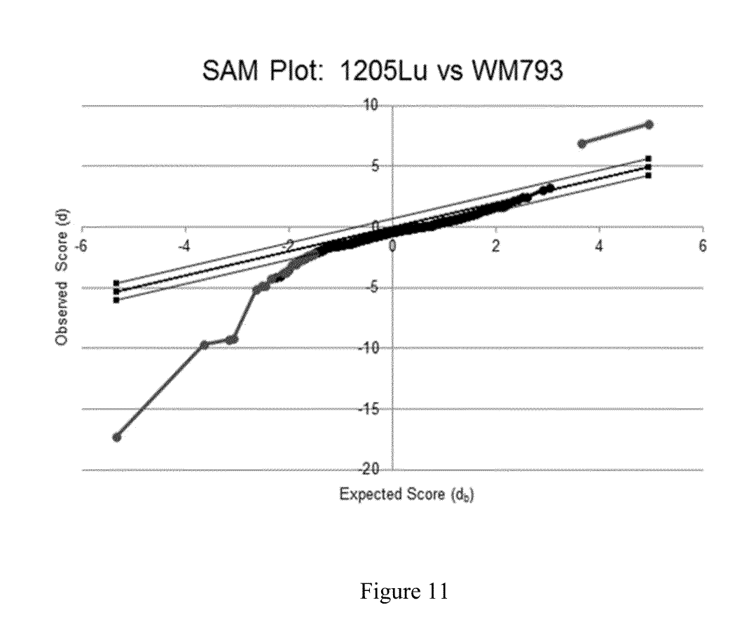

MS signal intensities for proteins identified in the combined media and interstitial fluid secretomes were analyzed by significance analysis of microarrays (SAM) (15) to identify proteins differentially secreted across high and low autophagy cell lines. Only proteins that were both significantly different and showed at least a 2-fold difference between 1205Lu and WM973 3D secretomes were considered as potentially meaningful differences (FIG. 11). Overall, these two secretomes were highly similar, verifying the utility of using genetically paired cell lines. Specifically, of the 599 proteins identified in this study, 571 were present at similar levels in both cell lines, 26 proteins were elevated in 1205Lu and two proteins were elevated in WM793 (FIG. 2A, Table 1). The estimated SAM false discovery rate was 3.5%.

Table 1 illustrates differentially secreted proteins across high and low autophagy melanoma cells.

TABLE 1

| | | | | | Fold |
|---|---|---|---|---|---|
| UniProtKB | | | | | Change |
| Accession | Gene | | Mean Intensity | | 1205Lu/ |
| ID | Name | Protein Name | 1205Lu | WM793 | WM793 |
| Q16769 | QPCT | Glutaminyl-peptide | 4.30 × 10⁶ | 2.79 × 10⁵ | 15 |
| | | cyclotransferase | | | |
| P10145-2 | IL8 | Isoform 2 of Interleukin 8 | 1.64 × 10⁶ | 1.19 × 10⁵ | 14 |
| P15018 | LlF | Leukemia inhibitory factor | 6.90 × 10⁶ | 5.20 × 10⁵ | 13 |
| O00560 | SDCBP | Syntenin-1 | 6.00 × 10⁵ | 4.75 × 10⁴ | 13 |
| P01584 | IL 1B | Interleukin-1 beta | 1.19 × 10⁵ | 9.62 × 10³ | 12 |
| C9JF17 | APOD | Apolipoprotein | 6.72 × 10⁶ | 5.50 × 10⁵ | 12 |
| Q92520 | FAM3C | Protein FAM3C | 4.77 × 10⁶ | 4.76 × 10⁵ | 10 |
| Q16610-4 | ECM1 | Extracellular matrix protein 1 | 1.14 × 10⁷ | 1.2S × 10⁶ | 9.1 |
| Q9UBP4 | DKK3 | Dickkopf-related protein 3 | 1.92 × 10⁶ | 2.19 × 10⁵ | 8.8 |
| P04083 | ANXA1 | Annexin A1 | 1.20 × 10⁵ | 2.11 × 10⁴ | 5.7 |
| B2R699 | GM2A | GM2 ganglioside activator | 8.82 × 10⁴ | 1.58 × 10⁴ | 5.6 |
| | | Protein | | | |
| O00468 | AGRN | Agrin precursor | 1.0S × 10⁶ | 2.11 × 10⁵ | 5.0 |
| P16035 | nMP2 | Metalloproteinase Inhibitor 2 | 2.90 × 10⁶ | 6.40 × 10⁵ | 4.5 |
| P15121 | AKR1B1 | Aldose Reductase | 1.54 × 10⁷ | 3.77 × 10⁶ | 4.1 |
| B3KQF4 | TIMP1 | Metalloproteinase inhibitor 1 | 5.04 × 10⁷ | 1.25 × 10⁷ | 4.0 |
| P22314 | UBE1 | Ubiquitin-like modifier- | 2.85 × 10⁶ | 7.42 × 10⁵ | 3.8 |
| | | activating enzyme 1 | | | |
| UPI0000445E06 | ACTI2 | Actin-like protein 2 | 8.12 × 10⁴ | 2.22 × 10⁴ | 3.7 |
| P07942 | LAMB1 | Laminin subunit beta-1 | 2.66 × 10⁷ | 7.82 × 10⁶ | 3.4 |
| P01034 | CST3 | Cystatin-C | 1.21 × 10⁶ | 3.68 × 10⁵ | 3.3 |
| Q2TU84 | GIG18 | Aspartate Aminotransferase | 3.75 × 10⁵ | 119 × 10⁵ | 3.2 |
| P45877 | PPIC | Peptidyl Prolyl cis trans | 7.46 × 10⁵ | 2.44 × 10⁵ | 3.1 |
| | | isomerase C | | | |
| Q14914 | PTGR1 | NADP-dependent leukotriene | 4.02 × 10⁵ | 1.43 × 10⁵ | 2.8 |
| | | B412-hydroxydehydrogenase | | | |
| A8K061 | ANGPnL3 | ANGPTL3 protein | 1.24 × 10⁶ | 4.65 × 10⁵ | 2.7 |
| P08670 | VIM | Vimentin | 7.09 × 10⁶ | 264 × 10⁶ | 2.7 |
| Q59GM9 | GOT1 | Phosphorylase | 3.11 × 10⁵ | 1.1S × 10⁵ | 2.7 |
| Q16674 | MIA | Melanoma-derived grow 1 h reg. | 1.44 × 10⁶ | 6.20 × 10⁵ | 2.3 |
| | | protein | | | |
| A6NII8 | LCN2 | LCN2 | 5.33 × 10⁶ | 1.85 × 10⁷ | −3.5 |
| Q9NRR1 | CYTL1 | Cytokine-like protein 1 | 4.12 × 10⁴ | 2.68 × 10⁵ | −6.6 |

Differentially secreted proteins were classified by Gene Ontology terms for biological processes and cellular components using the Software Tool for Rapid Annotation of Proteins (STRAP). In terms of subcellular localization, 32% of proteins were identified as extracellular matrix proteins, 16% were annotated as cytoplasmic, and another 11% corresponded to nuclear proteins (FIG. 2B). The remaining categories included plasma membrane, macromolecular complexes, cytoskeleton, peroxisome, and mitochondria, while 16% remained unclassified. Taken together, 38% of the differentially secreted proteins reside in the extracellular matrix, cell surface, or plasma membrane, the most common localizations previously identified for non-classically secreted proteins¹⁶. Non-classically secreted proteins also include cytokines, hormones, and growth factors that regulate cell growth, differentiation, invasion, and angiogenesis¹⁷, cell processes that represented 27% of the biological process GO terms derived from our secretome when including immune-related, developmental, and stimulus response processes (FIG. 2C).

To investigate molecular signaling pathways that may link subsets of differentially secreted proteins, a network map of these secreted proteins was constructed using the shortest paths algorithm based on functional relationships from the STRING database. The high confidence network interactions (probability >0.9) in our study represent functional linkages between proteins based on curated pathway and co-expression information. Betweenness-centrality clustering revealed a subnetwork highly centralized about the epidermal growth factor (EGF) node (FIG. 2D). Although the role of EGF remains controversial in melanoma, at least one group has determined EGF signaling contributes to lymph node metastasis¹⁸. Interestingly, network analysis identified a direct connection between EGF with the robustly elevated inflammatory cytokines leukemia inhibitory factor (LIF) and interleukin-1 beta (IL-1β) in high autophagy 1205Lu secretomes. The cytokine LIF has previously been shown to increase expression of interleukin-8 (IL8) and IL-1β, modulating inflammation and cancer progression¹⁹⁻²². Key components of this sub-network also included vimentin and TIMP2, both of which can promote cell motility through cytokine-dependent mechanisms²³⁻²⁴.

FIG. 2E illustrates a densely connected sub-network with the highly centralized glutaminyl peptide cyclotransferase (QPCT) node, which interacts with CTNNBL1, or beta-catenin, a key component of the oncogenic Wnt signaling pathway²⁵. The indirect functional connection of QPCT with AKR1B1 (aldo-keto reductase 1) via CTNNBL1 highlights the important functional relationship between Wnt signaling and inflammation²⁶. AKR1B1, which exhibited high centrality in inventors' network, forms a direct linkage with ANXA1, both of which can promote cell proliferation through NFκB-dependent inflammatory pathways²⁷⁻²⁹. In summary, this network analysis reveals linkages related to oncogenic and cytokine-mediated cell signaling, providing insights into the functional relevance of autophagy-associated secretions in melanoma.

### Example 5

### Evaluation of Selected Differentially Secreted Proteins as Potential Autophagy-Related Biomarkers in Melanoma

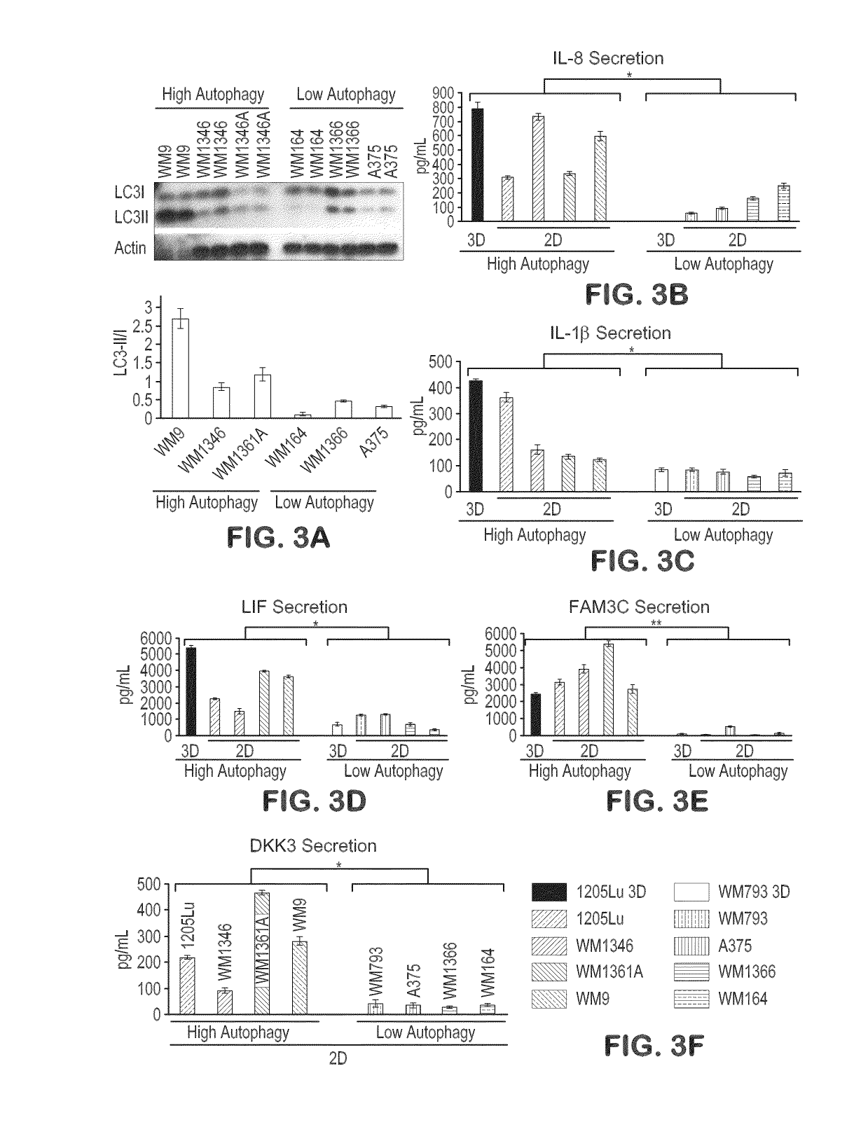

Commercially-available sandwich ELISA assays were used to evaluate the following biomarkers. These proteins were selected based on: 1) a minimum 6-fold increase in protein abundance in the 1205Lu secretome relative to the WM973 secretome (Table 1); 2) known involvement of the candidate in autophagic or tumorigenic networks, and 3) availability of high throughput assays for quantitation. The five proteins that met these criteria were IL-1β, IL-8, LIF, FAM3C, and Dickkopf-related Protein 3 (DKK3). Conditioned media from an independent set of melanoma cell lines with known autophagy levels were used to evaluate the generalizability of linkage to autophagy for these prioritized candidate biomarkers. Basal cellular autophagy was confirmed through LC3 immunoblotting (FIG. 3A), which verified that three additional cell lines exhibited relatively high autophagy (WM9, WM1361A, and WM1346), and three were low autophagy (WM1366, WM164, A375) cell lines. Conditioned media from these high and low autophagy cells as well as 1205Lu and WM793 were collected, and these biomarker levels were quantitated using ELISA. All five of these biomarkers showed a statistically significant elevation in media from the high autophagy cell lines, thereby confirming the results from our proteomics analysis (FIG. 3B).

Among these biomarkers, FAM3C showed the largest and most consistent increase in conditioned media from high autophagy cells with an average of ˜50-fold elevation. FAM3C is a cytokine that is associated with the epithelial to mesenchymal transition whose localization in the cytoplasm is associated with poor differentiation and prognosis in breast cancer³⁰.

### Example 6

### Effects of Tat-Beclin 1 on WM793 Autophagy and Secretion of Candidate Biomarkers

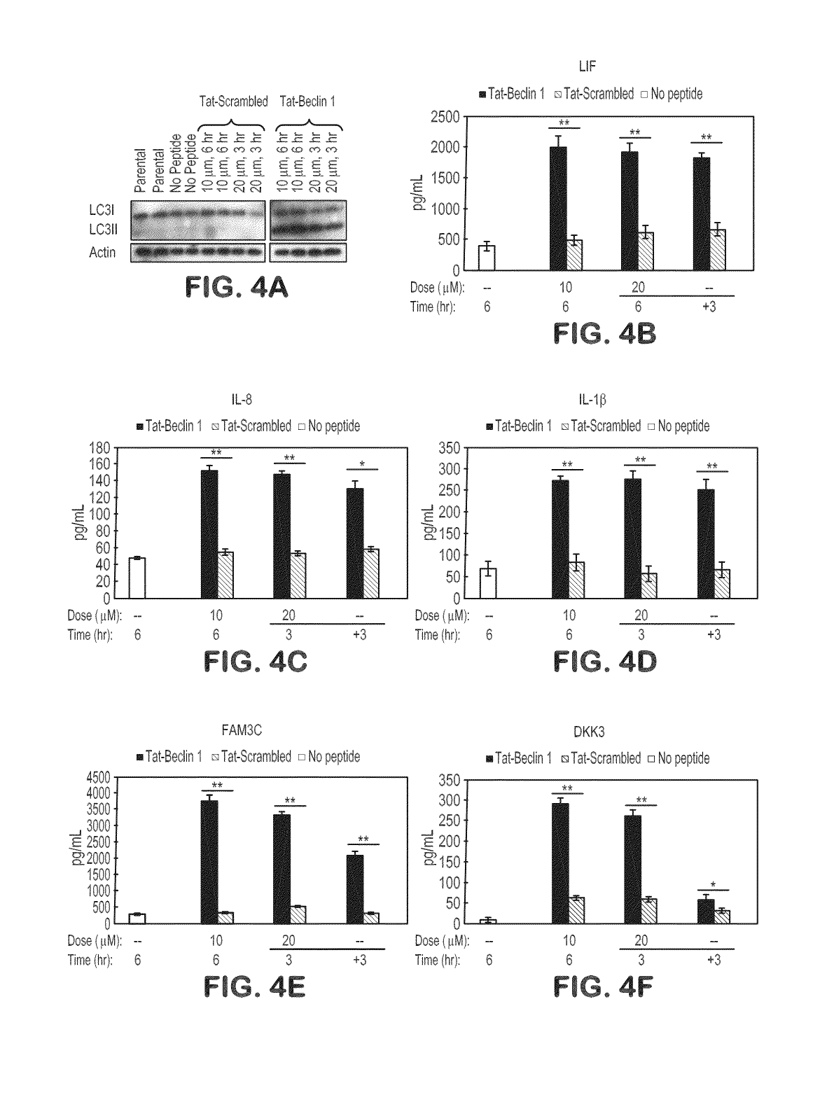

To more directly evaluate the autophagy dependence of candidate biomarker secretion, the low autophagy WM793 cell line was treated with Tat-Beclin 1, an autophagy inducing peptide composed of the HIV-1 Tat protein transduction domain and the amino acid sequence derived from the Nef-interacting domain of Beclin 1, an essential autophagy protein³¹. Tat-Scrambled, a peptide in which the 18 amino acids derived from the Beclin 1 sequence are randomly shuffled, was included as a control. Previous studies found that Tat-Beclin 1 induces a complete cellular autophagy response via the canonical autophagy pathway³¹. In WM793 cells, Tat-Beclin 1 treatment using 10 μM for 6 hours or 20 μM for 3 hours induced a potent autophagy response as indicated by the substantial increase in conversion of LC3I to LC3-II, compared with parental and control samples (FIG. 4A).

To determine the effects of targeted autophagy induction via Tat-Beclin 1 on the secretion of the candidate autophagy biomarkers, ELISA's were performed on conditioned media derived from cells treated without peptide, with Tat-Beclin 1, and with Tat-Scrambled control peptide (10 μM 6-hour and 20 μM 3 hour). Treatment with Tat-Beclin 1 induced a 3-4 fold increased rate of secretion versus control peptide and no peptide treatments in IL-8, IL-1β, and LIF, and up to a 10-fold increase in secretion of DKK3 and FAM3C (FIG. 4B). These results strongly support an autophagosome-dependent secretion mechanism for these five protein markers.

### Example 7

### Effects of Atg7 Silencing on WM1346 Autophagy and Secretion of Candidate Biomarkers

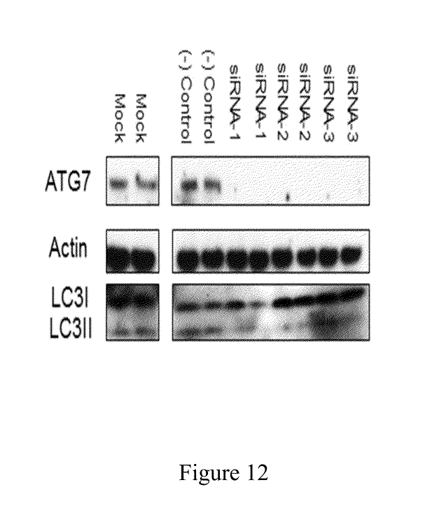

To further elucidate the role of autophagy in the secretion of the candidate biomarkers, siRNA-induced silencing of Atg7 was performed in the WM1346 melanoma cell line, which has a relatively high level of basal autophagy (FIG. 3A). Using three distinct sequences, Atg7 expression was effectively silenced with 20 nM siRNA, and for two of the three siRNA duplexes, a decrease in LC3-II conversion concomitant with Atg7 knockdown was observed (FIG. 12).

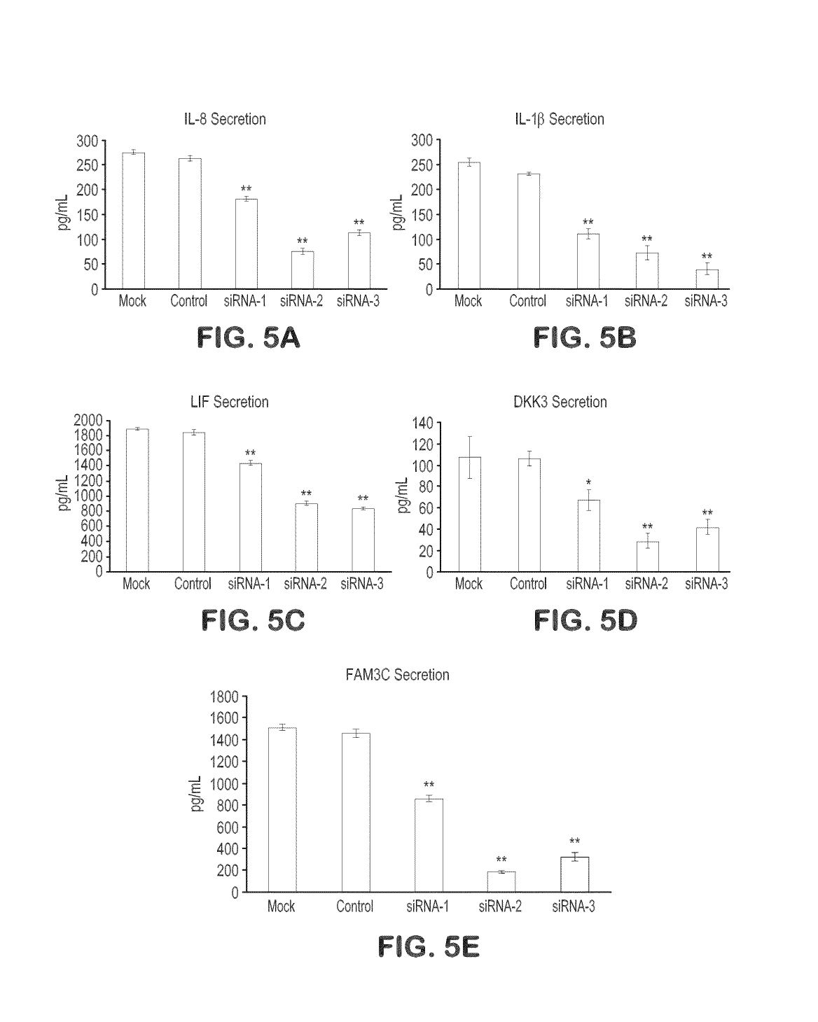

To determine whether Atg7 silencing decreased the secretion of these biomarker candidates, the conditioned media of cells treated with Atg7 siRNA duplexes vs. control and mock transfections were collected and probed for IL-8, IL-1β, LIF, DKK3, and FAM3C using ELISA. With Atg7 silencing, the levels of these proteins decreased significantly relative to controls (FIG. 5), underscoring a mechanistic linkage between autophagy levels and secretion of these proteins.

### Example 8

### Preliminary Clinical Evaluation of Candidate Autophagy Biomarkers in Melanoma Patient Serum

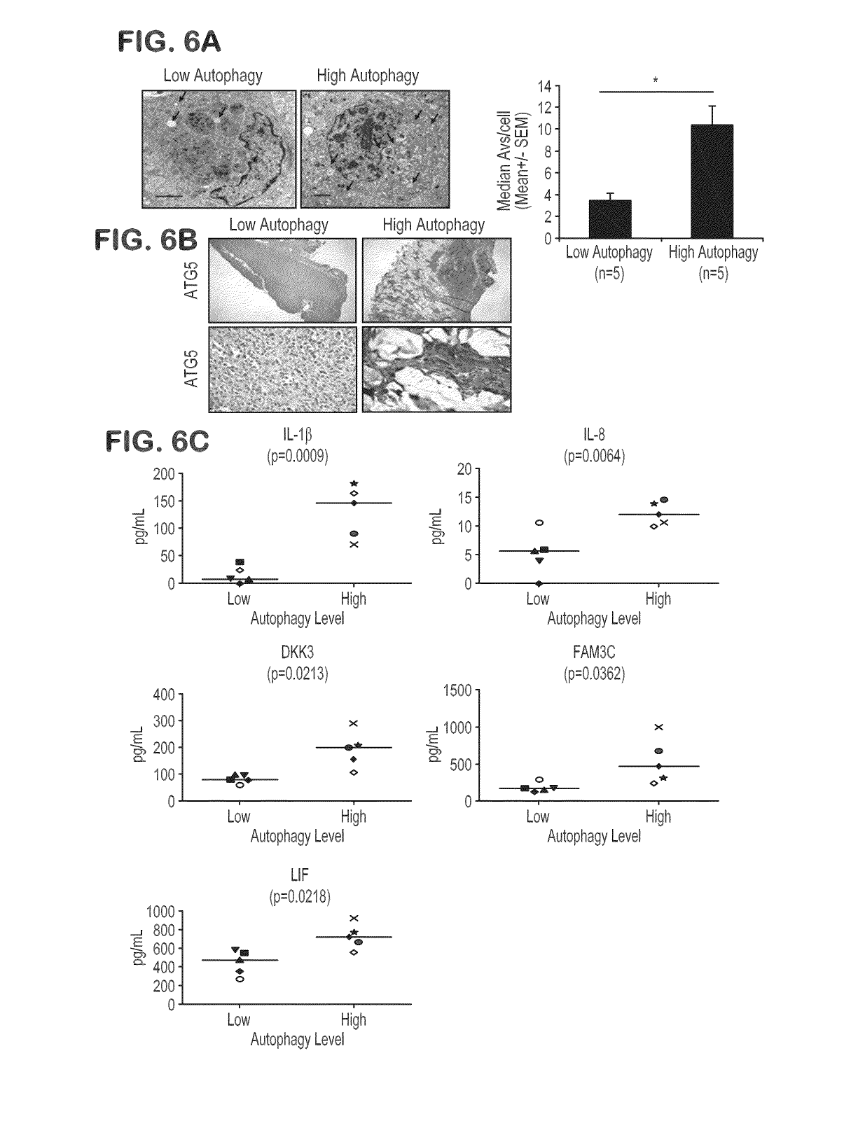

The five candidate biomarkers were subsequently assessed for potential association with autophagy in a small panel of serum samples derived from therapy-naïve melanoma patients, where autophagy levels in biopsied tumors were measured using quantitative electron microscopy (FIG. 6A). Patients whose tumors were found to have a mean number of AV/cell >6 were categorized as high autophagy tumors. Immunohistochemical staining for Atg5, an essential autophagy protein, in melanoma tumor tissue illustrated a robust elevation in Atg5 expression in the high autophagy subset of tumors, consistent with the EM results (FIG. 6B³²). The five candidate biomarkers were analyzed by sandwich ELISA in serum collected from these patients at the time of biopsy. Consistent with the proteomics and cell line validation analyses described above, these five proteins were significantly elevated in the serum from patients with high autophagy tumors (FIG. 6C).

Particularly striking was the ˜15-fold elevation in mean serum level of IL-1β in patients with high autophagy tumors. Interestingly, IL-1β has long been known as a protein that is non-classically secreted via an autophagy-based mechanism¹¹. The robust difference observed in these serum samples, combined with its well-characterized secretory phenotype, suggests that IL-1β may be among the more pivotal markers of high autophagy melanomas and therapy resistant melanomas.

Both the ATG7 knockdown experiments and the Tat-beclin 1 experiments described above support a mechanistic link between cellular autophagy levels and shedding the above-identified biomarkers into conditioned media.

As all five of the specified biomarkers IL-8, IL-1β, DKK-3, FAM-3C, and LIF tested in additional cell lines, patient sera, tat-beclin 1-based autophagy stimulation and ATG7 knockdown experiments correlated with tumor autophagy levels, it is reasonably anticipated that additional biomarkers having similar fold changes will behave similarly, such as QPCT, SDCBP, APOD and ECM1.

It is anticipated that testing of the biomarkers and biomarkers sets described herein in larger cohorts of patients collected from different sites, longitudinal prediagnostic blood specimens, and specimens collected throughout therapeutic treatment will demonstrate results consistent with the above. For instance, these candidate biomarkers are anticipated to be useful in predicting response to autophagy inhibitors and other autophagy targeting therapies. A similar approach will be undertaken to characterize autophagy dynamics in other tumors, which are likely to have different but possibly overlapping autophagy-dependent secretome signatures.

Each and every patent, patent application, and publication, including publications listed below, and each publically available nucleotide, oligonucleotide and protein sequences cited throughout the disclosure, is expressly incorporated herein by reference in its entirety. Embodiments and variations of this invention other than those specifically disclosed above may be devised by others skilled in the art without departing from the true spirit and scope of the invention. The appended claims include such embodiments and equivalent variations.

### PUBLICATIONS

1. X. H. Ma, et al. Measurements of tumor cell autophagy predict invasiveness, resistance to chemotherapy, and survival in melanoma. Clin Cancer Res. 17, 3478-89 (2011).
2. B. Levine, G. Kromer. Autophagy in the pathogenesis of disease. Cell. 132, 27-42 (2008).
3. R. K. Amaravadi et al, Principles and current strategies for targeting autophagy for cancer treatment. Clin Cancer Res. 17, 654-66 (2011).
4. Y. Rong, et al (2011). Spinster is required for autophagic lysosome reformation and mTOR reactivation following starvation. Proc Natl Acad Sci USA. 108(19): 7826-31.
5. R. Lazova, et al. Punctate LC3B expression is a common feature of solid tumors and associated with proliferation, metastasis, and poor outcome. Clin Cancer Res. 18, 370-9 (2012).
6. A. K. Ganesan, et al. Genome-wide siRNA-based functional genomics of pigmentation identifies novel genes and pathways that impact melanogenesis in human cells. PLoS Genet. 4, e1000298 (2008).
7. J. A. Sosman, et al. Survival in BRAF V600-mutant advanced melanoma treated with vemurafenib. N Engl J Med. 366, 707-714 (2012).
8. R. D. Leone, R. K. Amaravadi. Autophagy: a targetable linchpin of cancer cell metabolism. Trends Endocrinol Metab. 24, 209-17 (2013).
9. S. Barth, et al. Autophagy: assays and artifacts. J Pathol. 221, 117-24 (2010).
10. W. Martinet, et al. In Situ Detection of Starvation-induced Autophagy. J Histochem Cytochem. 54, 85-96 (2006).
11. N. Dupont, et al. Autophagy-based unconventional secretory pathway for extracellular delivery of IL-1β. EMBO J. 30, 4701-11 (2011).
12. M. Narita, et al. Spatial coupling of mTOR and autophagy augments secretory phenotypes. Science. 332, 966-70 (2011).
13. C. J. DeSelm, et al. Autophagy proteins regulate the secretory component of osteoclastic bone resorption. Dev Cell. 21, 966-74 (2011).
14. I. Juhasz, et al. Growth and Invasion of human melanomas in human skin grafted to immunodeficient mice. Am J Pathol. 143, 528-37 (1993).
15. B. A. Roxas, Q. Li. Significance analysis of microarray for relative quantitation of LC/MS data in proteomics. BMC Bioinformatics. 9, 187-203 (2008).
16. J. D. Bendtsen et al. Feature-based prediction of non-classical and leaderless protein secretion. Protein Eng Des Sel. 17, 349-56 (2004).
17. P. Dowling, M. Clynes. Conditioned media from cell lines: a complementary model to clinical specimens for the discovery of disease-specific biomarkers. Proteomics. 11, 794-804 (2011).
18. A. Bracher, et al. Epidermal growth factor facilitates melanoma lymph node metastasis by influencing tumor lymphangiogenesis. J Invest Dermatol. 133, 230-8 (2013).
19. P. M. Villiger, et al. Induction of cytokine expression by leukemia inhibitory factor. J Clin Invest. 91, 1575-81 (1993).
20. D. J. Waugh, C. Wilson. The interleukin-8 pathway in cancer. Clin Cancer Res. 14, 6735-41 (2008).
21. J. Harris. Autophagy and Cytokines. Cytokine. 56, 140-44 (2011).
22. E. Voronov, et al. IL-1 is required for tumor invasiveness and angiogenesis. Proc Natl Acad Sci USA. 100, 2645-50 (2003).
23. A. Satelli, S. Li. Vimentin in cancer and its potential as a molecular target for cancer therapy. Cell Mol Life Sci. 68, 3033-46 (2011).
24. C. Ries, et al. MMP-2, MT1-MMP, and TIMP-2 are essential for the invasive capacity of human mesenchymal stem cells: differential regulation by inflammatory cytokines. Blood. 109, 4055-63 (2007).
25. Y. Kawano, R. Kypta. Secreted antagonists of the Wnt signaling pathway. Journal of Cell Science. 116, 2627-2634 (2003).
26. B. Gustafson, U. Smith. Cytokines promote Wnt signaling and inflammation and impair the normal differentiation and lipid accumulation in 3T3-L1 preadipocytes. J Biol Chem. 281, 9507-16 (2006).
27. B. Laffin, J. M. Petrash. Expression of the Aldo-Ketoreductases AKR1B1 and AKR1B10 in Human Cancers. Front Pharmacol. 3, 104 (2012).
28. K. V. Ramana, et al. Aldose reductase-regulated tumor necrosis factor-alpha production is essential for high glucose-induced vascular smooth muscle cell growth. Endocrin, 148, 4371-84 (2007).
29. Z. Zhang, et al. Annexin 1 induced by anti-inflammatory drugs binds to NF-kappaB and inhibits its activation: anticancer effects in vitro and in vivo. Cancer Res. 70, 2379-88 (2010).
30. T. Waerner, et al. ILEI: a cytokine essential for EMT, tumor formation, and late events in metastasis in epithelial cells. Cancer Cell. 10, 227-39 (2006).
31. S. Shoji-Kawata, et al. Identification of a candidate therapeutic autophagy-inducing peptide. Nature. 494, 201-6 (2013).
32. J. O. Pyo, et al. Overexpression of Atg5 in mice activates autophagy and extends lifespan. Nat Commun 4, 2300 (2013).
33. R. Manjithaya, S. Subramani. Role of autophagy in unconventional protein secretion. Autophagy. 6, 650-1 (2010).
34. M. Okamoto, et al. Constitutively active inflammasome in human melanoma cells mediating autoinflammation via caspase-1 processing and secretion of interleukin-1beta. J Biol Chem. 285, 6477-88 (2010).
35. B. Levine, et al. Autophagy in immunity and inflammation. Nature. 469, 323-35 (2011).
36. B. G. Byrne, et al. Inflammasome components coordinate autophagy and pyroptosis as macrophage responses to infection. MBio. 4, e00620-12 (2013).
37. L. Zitvogel, et al. Inflammasomes in carcinogenesis and anticancer immune responses. Nat Immunol. 13, 343-51 (2012).
38. R. K. Singh, M. L. Varney. Regulation of interleukin 8 expression in human malignant melanoma cells. Cancer Res. 58, 1532-7 (1998).
39. M. Luca, et al. Expression of interleukin-8 by human melanoma cells up-regulates MMP-2 activity and increases tumor growth and metastasis. Am J Pathol. 151, 1105-13 (1997).
40. E. M. Salas, et al. LIF, a Novel STATS-Regulated Gene, Is Aberrantly Expressed in Myeloproliferative Neoplasms. Genes Cancer. 2, 593-6 (2011).
41. S. Maruta, et al. A role for leukemia inhibitory factor in melanoma-induced bone metastasis. Clin Exp Metastasis. 26, 133-41 (2009).
42. C. Lahsnig, et al. ILEI requires oncogenic Ras for the epithelial to mesenchymal transition of hepatocytes and liver carcinoma progression. Oncogene. 28, 638-50 (2009).
43. W. Yue, et al. Downregulation of Dkk3 activates β-catenin/TCF-4 signaling in lung cancer. Carcinogenesis. 29, 84-92 (2007).
44. F. Abarzua, et al. Adenovirus-mediated overexpression of REIC/Dkk-3 selectively induces apoptosis in human prostate cancer cells through activation of c-Jun-NH2-kinase. Cancer Research. 65, 9617-22 (2005).
45. Z. R. Yang, et al. Overexpression of Dickkopf-3 induces apoptosis through mitochondrial pathway in human colon cancer. World Journal of Gastroenterology. 18, 1590-601 (2012).
46. J. Veeck, E. Dahl. Targeting the Wnt pathway in cancer: the emerging role of Dickkopf-3. Biochim Biophys Acta. 1825, 18-28 (2012).
47. G. Untergasser, et al. The Dickkopf-homolog 3 is expressed in tumor endothelial cells and supports capillary formation. Int J Cancer. 122, 1539-47 (2008).
48. L. A. Beer, et al. Systematic discovery of ectopic pregnancy serum biomarkers using 3-D protein profiling coupled with label-free quantitation. J Proteome Res. 10, 1126-38 (2011).
49. E. N. Pesciotta, et al. A label-free proteome analysis strategy for identifying quantitative changes in erythrocyte membranes induced by red cell disorders. J Proteomics. 76, 194-202 (2012).
50. U. auf dem Keller, et al. Overall. A statistics-based platform for quantitative N-terminome analysis and identification of protease cleavage products. Mol Cell Proteomics. 9, 912-27 (2010).
51. M. Baitaluk, et al. Biological Networks: visualization and analysis tool for systems biology. Nucleic Acids Res. 34, 466-71 (2006).
52. M. Baitaluk, et al. IntegromeDB: an integrated system and biological search engine. BMC Genomics. 13, 35 (2012).
53. R. K. Amaravadi, et al. Phase II Trial of Temozolomide and Sorafenib in Advanced Melanoma Patients with or without Brain Metastases. Clin Cancer Res. 15, 7711-7718 (2009).

1. A diagnostic reagent, kit or device comprising two or more ligands selected from:
(a) a ligand capable of specifically complexing with, binding to, or quantitatively detecting or identifying IL-8,
(b) a ligand capable of specifically complexing with, binding to, or quantitatively detecting or identifying IL-1β,
(c) a ligand capable of specifically complexing with, binding to, or quantitatively detecting or identifying DKK-3,
(d) a ligand capable of specifically complexing with, binding to, or quantitatively detecting or identifying FAM-3C,
(e) a ligand capable of specifically complexing with, binding to, or quantitatively detecting or identifying LIF,
(f) a ligand capable of specifically complexing with, binding to, or quantitatively detecting or identifying an isoform, pro-form, modified molecular form, posttranslational modification, or unique peptide fragment or unique nucleic acid fragment of any of (a) through (e), or
(g) a ligand capable of specifically complexing with, binding to, or quantitatively detecting or identifying a protein in the same biomarker family or expressed from a related gene having at least 20% sequence homology or sequence identity with any biomarker (a) to (e),
wherein one or more of said ligands (a) to (g) is covalently or noncovalently joined to a molecule or moiety capable alone or in combination with one or more additional molecules of generating a detectable signal, or is immobilized on a substrate.

2. The reagent, kit or device according to claim 1, further comprising one or more additional ligands, each additional ligand capable of specifically complexing with, binding to, or quantitatively detecting, or identifying an additional biomarker that indicates the presence of melanoma in a human subject.

3. The reagent, kit or device according to claim 2, wherein the additional biomarker is a biomarker of Table 1 or an isoform, pro-form, modified molecular form, posttranslational modfication, or unique peptide fragment or unique nucleic acid fragment thereof.

4. The reagent, kit or device according to claim 1, comprising a set of two or more ligands, each ligand individually capable of specifically complexing with, binding to, or quantitatively detecting or identifying a single biomarker or an isoform, pro-form, modified molecular form, posttranslational modification, or unique peptide fragment or nucleic acid fragment thereof, said set of ligands identifying the biomarkers
(a) IL-8 and IL-1β,
(b) IL-8 and DKK-3,
(c) IL-8 and FAM-3C
(d) IL-8 and LIF
(e) IL-8, IL-1β and DKK-3,
(f) IL-8, IL-1β, and FAM-3C,
(g) IL-8, IL-1β, and LIF
(h) IL-8, DKK-3 and FAM-3C
(i) IL-8, DKK-3 and LIF
(j) IL-8, FAM-3C and LIF
(k) IL-8, DKK-3, FAM-3C and LIF
(l) IL-8, IL-1β, FAM-3C and LIF
(m) IL-8, IL-1β, DKK-3, and FAM-3C
(n) IL-8, IL-1β, DKK-3, and LIF
(o) IL-8, IL-1β, DKK-3, FAM-3C and LIF
(p) IL-1β and DKK-3,
(q) IL-1β and FAM-3C
(r) IL-1β and LIF
(s) IL-1β, DKK-3, and FAM-3C
(t) IL-1β, LIF and FAM-3C,
(u) IL-1β, DKK-3 and LIF
(v) IL-1β, DKK-3, FAM-3C and LIF
(w) DKK-3 and FAM-3C
(x) DKK-3 and LIF, or
(y) DKK-3, LIF, and FAM-3C.

5. The reagent, kit or device according to claim 1 comprising 2, 3, 4, or 5 ligands.

6. The reagent, kit or device according to claim 1, wherein any of said ligands comprises an antibody or fragment of an antibody, an antibody mimic, a synthetic antibody, a bi- or tri-specific antibody, a single chain antibody or any amino acid sequence that binds to or complexes with a single biomarker, said ligand optionally associated with a detectable label or with a substrate.

7. The reagent, kit or device according to claim 1, wherein any of said ligands comprises a nucleotide sequence capable of hybridizing to a nucleic acid sequence encoding a single biomarker, said ligand optionally associated with a detectable label or with a substrate.

8. The reagent, kit or device according to claim 1, wherein said substrate is a microarray, a microfluidics card, a glass slide, a chip, a bead, or a chamber.

9. The reagent, kit or device according to claim 1, which comprises a panel or microarray of said ligands or their biomarker targets.

10. A method for identifying the autophagy level of a cell in a biological sample comprising:
contacting a biological sample containing cells obtained from a test subject with a diagnostic reagent or device of claim 1;
measuring or detecting in the sample the complex formed by the reagent and a biomarker of claim 1 present in the sample, and obtaining the protein level of the biomarker;
determining a significant modulation in protein level of the biomarker of claim 1 in the subject's sample relative to a reference standard, wherein the modulation correlates with an increase or decrease in the autophagy level in the subject's cells.

11. The method according to claim 10, wherein the subject has a cancer or tumor.

12. The method according to claim 10, further comprising obtaining the sample by minimally invasive means.

13. The method according to claim 10, wherein the biological sample is selected from group consisting of whole blood, plasma, serum, circulating tumor cells, ascites fluid, peritoneal fluid and tumor tissue or tissue or fluid from a biopsy sample, surgical sample, or tumor cell sample.

14. The method according to claim 11, which is an enzyme-linked immunoassay.

15. An assay method for predicting the sensitivity of a subject with cancer to treatment with an autophagy inhibitor comprising
contacting a biological sample obtained from a test subject with a diagnostic reagent or device of claim 1; and
detecting or measuring in the sample the complex formed by the reagent and a biomarker of claim 1 in the sample, and generating a biomarker protein level therefrom;
comparing the protein level of the biomarker in the subject's sample with the level of the same biomarker in an temporally earlier sample from the subject, a subject's sample prior to treatment with the inhibitor, or a reference standard from healthy subjects;
wherein a significant increase in biomarker level in the subject's sample relative to that in the reference standard indicates the subject's response to the inhibitor.

16. The method according to claim 15, which is an enzyme-linked immunoassay.

17. An assay method for assessing the chemo-resistance of a cancer comprising contacting a biological sample obtained from a test subject with a diagnostic reagent or device of claim 1; and
detecting or measuring in the sample the complex formed by the reagent and a biomarker of claim 1 in the sample and generating the protein level of at least one biomarker of claim 1;
comparing the protein level of the biomarker in the subject's sample with the level of the same biomarker in an temporally earlier sample from the subject, either treated or untreated with a chemotherapeutic, a reference standard from subjects with cancer that is not chemoresistant, or a reference standard from healthy subjects;
wherein a significant increase in biomarker level in the subject's sample relative to that in the reference standards indicates the subject's cancer is chemoresistant.

18. A method for treating a subject with melanoma comprising:
contacting a biological sample obtained from a test subject with a diagnostic reagent or device of claim 1;
quantitatively measuring tumor autophagy levels in the subject by detecting or measuring in the sample the complex formed by the reagent and a biomarker of claim 1 and generating a protein level of the biomarker of claim 1;
comparing the protein level of the biomarker in the subject's sample with the level of the same biomarker in a healthy reference standard or in a reference standard of subjects with non-chemoresistant melanoma; and
treating said subject with an autophagy inhibitor, when the biomarker expression levels in the subject's tumor sample are increased over that of the reference standard.

19. A method for treating a subject with cancer comprising detecting in a blood sample of a subject a change in the expression level of a protein or peptide selected from one or more of IL-8, IL-1β, DKK-3, FAM-3C, and LIF, wherein the change is relative to a baseline expression level obtained from a previous blood sample from the sample subject or a reference standard;
diagnosing a cancer characterized by high autophagy levels, ineffective treatment of a cancer, or advance of a cancer, wherein an increase in expression of said protein or proteins is observed; and
treating said subject with an autophagy inhibitor.
